SEQUENCE LISTING (10 sequences)

SEQ ID NO: 1 - protein, 11 aa, synthetic construct
LXXLXLXXNXL

SEQ ID NO: 2 - protein, 12 aa, synthetic construct
LXXLXLXXNXXL

SEQ ID NO: 3 - protein, 8 aa, synthetic construct
GSGRSGSG

SEQ ID NO: 4 - protein, 30 aa, Homo sapiens
MGNTIRALVAFIPADRCQNYVVRDLREMPL

SEQ ID NO: 5 - protein, 184 aa, Homo sapiens
MGNTIRALVAFIPADRCQNYVVRDLREMPLDKMVDLSGSQLRRFPLHVCSFREGSGRSGSGQVQLQESGGGLVQAGGSLRLSCVASGRTFSSYAMGWFRQAPGKEREFVAAIDWSGGTASHADSVKGRFTISRDNAKNTVYLQMNSLKPEDTAVYYCAASSYWSRSVDEYDYWGQGTQVTVSSX

SEQ ID NO: 6 - DNA, 552 bp, Homo sapiens
  source 1..552: mol_type = genomic DNA
atggggaacaccatcagggccctcgtggccttcatccctgctgaccgttgccagaactatgtggtcagggacctccgtgagatgccgctggacaagatggtggatctgagtgggagccagttacgccgcttccccctgcacgtgtgctccttcagggagggaagtgggagatctggaagcgggcaggtgcagctgcaggagtctgggggaggattggtgcaggctgggggctctctgagactctcctgtgtagcctctggacgcaccttcagtagctatgccatgggctggttccgccaggctccagggaaggagcgtgagtttgtagcagctattgactggagtggtggtaccgcaagccatgcagactccgtgaagggccgattcaccatctccagagacaacgccaagaacacggtgtatctgcaaatgaacagcctgaaacctgaggacacggccgtttattactgtgcagcctcatcatattggtcgagatcggtagacgagtatgactactggggccaggggacccaggtcaccgtctcctcataa

SEQ ID NO: 7 - DNA, 36131 bp, synthetic construct
  source 1..36131: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggggaacaccatcagggccctcgtggccttcatccctgctgaccgttgccagaactatgtggtcagggacctccgtgagatgccgctggacaagatggtggatctgagtgggagccagttacgccgcttccccctgcacgtgtgctccttcagggagggaagtgggagatctggaagcgggcaggtgcagctgcaggagtctgggggaggattggtgcaggctgggggctctctgagactctcctgtgtagcctctggacgcaccttcagtagctatgccatgggctggttccgccaggctccagggaaggagcgtgagtttgtagcagctattgactggagtggtggtaccgcaagccatgcagactccgtgaagggccgattcaccatctccagagacaacgccaagaacacggtgtatctgcaaatgaacagcctgaaacctgaggacacggccgtttattactgtgcagcctcatcatattggtcgagatcggtagacgagtatgactactggggccaggggacccaggtcaccgtctcctcataaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacaactttattatacatagttgatggccggccgcttcgaggggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagcttttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcccgggagagctcgatatcgcatgcggatttaaattaattaa

SEQ ID NO: 8 - DNA, 5942 bp, synthetic construct
  source 1..5942: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctataacttcgtataggatactttatacgaagttatcctcgagtaccggtcacgcgtacgcaagcttgataacttcgtatagcatacattatacgaagttatcctagggccaccttacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccataggcccggggttttcttcaacatctcctgcttgctttaacagagagaagttcgtggctccgcttcctgaggagacggtgacctgggtcccctggccccagtagtcatactcgtctaccgatctcgaccaatatgatgaggctgcacagtaataaacggccgtgtcctcaggtttcaggctgttcatttgcagatacaccgtgttcttggcgttgtctctggagatggtgaatcggcccttcacggagtctgcatggcttgcggtaccaccactccagtcaatagctgctacaaactcacgctccttccctggagcctggcggaaccagcccatggcatagctactgaaggtgcgtccagaggctacacaggagagtctcagagagcccccagcctgcaccaatcctcccccagactcctgcagctgcacctgcccgcttccagatctcccacttccctccctgaaggagcacacgtgcagggggaagcggcgtaactggctcccactcagatccaccatcttgtccagcggcatctcacggaggtccctgaccacatagttctggcaacggtcagcagggatgaaggccacgagggccctgatggtgttccccatggtggctctagaataacttcgtataaagtatcctatacgaagttataggtacctgagctcaacatcgatagcactagtataacttcgtataatgtatgctatacgaagttatacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 9 - DNA, 35195 bp, synthetic construct
  source 1..35195: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcccgggagagctcgatatcgcatgcggatttaaattaattaa

SEQ ID NO: 10 - DNA, 5118 bp, synthetic construct
  source 1..5118: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

(19) United States
(12) Patent Application Publication
(10) Pub. No.: US 2026/0217774 A1
(43) Pub. Date: Jul. 30, 2026

(54) GENETICALLY ENCODED ENHANCER OF CALCIUM CURRENTS FOR TREATMENT OF CARDIAC, NEUROLOGIC, AND PSYCHIATRIC DISEASES

(71) Applicant: THE TRUSTEES OF COLUMBIA UNIVERSITY IN THE CITY OF NEW YORK, New York, NY (US)

(72) Inventors: Pedro Javier Del Rivero Morfin, New York, NY (US); Manu Ben-Johny, New York, NY (US)

(21) Appl. No.: 19/426,510

(22) Filed: Dec. 19, 2025

Related U.S. Application Data
(63) Continuation of application No. PCT/US2024/035919, filed on Jun. 27, 2024.
(60) Provisional application No. 63/511,584, filed on Jun. 30, 2023.
(60) Provisional application No. 63/646,731, filed on May 13, 2024.

(51) Int. Cl. C07K 14/435; A61K 38/00; A61P 25/00; C07K 16/18; C12N 15/86

(52) U.S. Cl. CPC C07K 14/435; A61P 25/00; C07K 16/18; C12N 15/86; A61K 38/00; C07K 2317/569; C07K 2319/30; C12N 2750/14143

(57) ABSTRACT

The present invention describes a fusion protein or peptide conjugate comprising the functional domain of a cardiac-specific short chain amino acid with low affinity for the Ca_{V}β-subunit conjugated. The fusion protein or peptide conjugate upregulates the function of L-type calcium channels in a cell-type dependent manner. In some aspects, the fusion protein is a chimeric genetically encoded enhancer of calcium channels (GeeC) comprising the functional domain of a cardiac-specific short chain amino acid with low affinity for the Ca_{V}β-subunit conjugated to a functionally inert nanobody with high affinity to the Ca_{V}β-subunit. In some aspects, GeeC enables targeted upregulation of Ca_{V}1.2/1.3 channels in diverse physiological settings thereby opening new avenues to development of future therapeutics. Methods for creating and administering the GeeC to human and non-human animals for the purposes of experimenting and treating cardiac, neurological, and psychiatric pathologies are also described.

### CROSS-REFERENCE TO RELATED APPLICATIONS

This application is a continuation of International Application No. PCT/US2024/035919, filed on Jun. 27, 2024, which claims priority to U.S. Provisional Patent Application No. 63/511,584 filed on Jun. 30, 2023, and U.S. Provisional Patent Application No. 63/646,731 filed on May 13, 2024. The present application is filed under 35 U.S.C. § 111 (a) as a U.S. bypass continuation application of the above-referenced PCT application. The entireties of the aforementioned applications are hereby incorporated by reference.

### STATEMENT REGARDING FEDERALLY SPONSORED RESEARCH OR DEVELOPMENT

This invention was made with government support under HL164319 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 92,731 byte XML file named “SeqList” created on Jun. 26, 2024.

### BACKGROUND OF THE INVENTION

L-type calcium (Ca²⁺) channels (LTCCs) are biologically crucial as they convert electrical signals to Ca²⁺ influx, which orchestrates several physiological processes. These include: (1) shaping the action potential (AP), and initiating excitation-contraction coupling in the heart; (2) tuning neuronal excitability and excitation-transcription coupling that contributes to synaptic plasticity in neurons; (3) enabling insulin secretion in pancreatic beta cells; (4) regulating AP firing and catecholamine release from adrenal chromaffin cells; (5) supporting smooth muscle contraction; (6) triggering vesicle secretion in inner ear hair cells; and (7) varied functions in non-excitable cells. Thus, LTCCs serve as vital portals for Ca²⁺ entry into cardiac myocytes, neurons, and other physiological systems.

Altered LTCC function is linked to various pathologies including cardiac arrhythmias and neurodevelopmental disorders. In particular, genetic studies have identified loss-of-function mutations in Ca_{V}1.2 channels in patients with cardiac arrythmias, shortened QT interval, and Brugada Syndrome, as well as various neurological phenotypes, including neuropsychiatric disorders. Gain-of-function Ca_{V}1.2 variants are linked to a multisystem disorder, Timothy Syndrome.

Ca_{V}1.2 (CACNAIC) and/or Ca_{V}1.3 (CACNAID) channels have been found to be prominent risk genes for various neurodevelopmental and neuropsychiatric disorders including ASD, schizophrenia, and bipolar disorder, suggesting important yet complex functions in the brain. In neurons, Ca²⁺ influx after depolarization contributes to excitation-transcription coupling (E-T coupling), a key cellular mechanism where elevated intraneuronal Ca²⁺ modulates transcription of select genes, integrating electrical signals from neurons with protein synthesis. Altered LTCC activity in neurons is associated with several neuropsychiatric diseases where loss of function mutations in the Ca_{V}1.2 subtype of LTCC results in reduced protein synthesis in the prefrontal cortex and loss of Ca_{V}1.3 subtype disrupts proliferation of neuron progenitors and compromises the survival of newborn hippocampal neurons. These pathophysiological features translate into altered behaviors like anxiety-related symptoms and memory impairment.

In the heart, LTCCs contribute to shaping the action potential, conveying Ca²⁺ into the cell to initiate excitation contraction coupling. During adrenergic stimulation, increased Ca²⁺ entry into cardiomyocytes improves inotropy, resulting in enhanced heart contraction and ejection fraction. Alterations in Ca²⁺ influx into cardiomyocytes, as well as imbalances in basal Ca²⁺, lead to cardiac remodeling during heart failure and to potentially lethal cardiac arrhythmia like short QT syndrome.

Given the broad physiological distribution and importance of LTCC in various tissues, conventional pharmacological agonists of LTCC can yield off-target effects and toxicity. Therefore, new approaches to upregulate LTCC function in a cell-type dependent manner are sought by those in the field.

### SUMMARY OF THE INVENTION

A fusion protein or peptide conjugate that enhances Ca_{V}1.2 and Ca_{V}1.3 channel activity and L-type currents as well as related compositions are described herein. The fusion protein or peptide conjugate comprises polypeptide derived from or is a portion of a leucine-rich repeat-containing protein 10 (LRRC10) and a targeting agent selective for a β subunit of a L-type Ca²⁺ channel (LTCC). The targeting agent is conjugated to the polypeptide. The polypeptide modulates channel activity of Ca_{V}1.2 and/or Ca_{V}1.3.

In some aspects, the polypeptide comprises the amino-terminus (NT domain) of LRRC10 or a portion thereof. For example, the polypeptide comprises the first 53 amino acids of LRRC10. In some embodiments, the polypeptide has an amino acid sequence comprising less than 60 amino acids. In certain embodiments, the polypeptide is derived from or is a portion of human LRRC10.

In some aspects, the targeting agent is selective for a β subunit of Ca_{V}1.2 or Ca_{V}1.3. In some embodiments, the targeting agent has a greater binding affinity to a Ca_{V}β subunit than the polypeptide. In some embodiments of the fusion protein, the targeting agent is a nanobody, for example, nb.F3.

In certain embodiments, the amino acid sequence of the fusion protein is set forth in SEQ ID NO. 5. In particular embodiments, the nucleic acid sequence encoding the fusion protein is set forth in SEQ ID NO. 6.

In some aspects, pharmaceutical compositions comprising the fusion protein or peptide conjugate are disclosed, for example for use in enhancing Ca_{V}1.2 and Ca_{V}1.3 channel activity in a cell or enhancing L-type currents in a cell or in treating Rett Syndrome (RTT). In other aspects, pharmaceutical compositions comprising a nucleic acid sequence encoding the fusion protein are disclosed. In such embodiments, the pharmaceutical composition comprises an adenovirus vector or comprises an adenovirus. In compositions comprising the adenovirus vector, the adenovirus vector comprises the nucleic acid sequence encoding the fusion protein. In composition comprising the adenovirus, the adenovirus has been transfected with an adenovirus vector that comprises the nucleic acid sequence encoding the fusion protein.

A method of upregulating Ca_{V}1.2 and/or Ca_{V}1.3 current in a cell is also disclosed herein. The method comprises administering the fusion protein disclosed herein to the cell. In some implementations, the fusion protein comprises a polypeptide derived from LRRC10 and a targeting agent, where the targeting agent is conjugated to the polypeptide. The targeting agent is selective for a β subunit of a L-type Ca²⁺ channel (LTCC), and the polypeptide comprises the NT domain of LRRC10 or a portion thereof. In some embodiments, the method comprises transducing the cell with an adenovirus, wherein the adenovirus comprises a nucleic acid sequence encoding the fusion protein and thus results in the administration of the fusion protein to the cell. In some implementations, the cell is a neuron or a muscle cell. In some aspects, the muscle cell is a cardiomyocyte.

A method of enhancing L-type currents in a cell is further disclosed herein. The method comprises administering the fusion protein disclosed herein to the cell. In some implementations, the fusion protein comprises a polypeptide derived from LRRC10 and a targeting agent, where the targeting agent is conjugated to the polypeptide. The targeting agent is selective for a β subunit of a L-type Ca²⁺ channel (LTCC), and the polypeptide comprises the NT domain of LRRC10 or a portion thereof. In some embodiments, the method comprises transducing the cell with an adenovirus, wherein the adenovirus comprises a nucleic acid sequence encoding the fusion protein and thus results in the administration of the fusion protein to the cell. In some implementations, the cell is a neuron or a muscle cell. In some aspects, the muscle cell is a cardiomyocyte.

A method of reducing neurological symptoms in a subject diagnosed with Rett Syndrome (RTT) is additionally disclosed herein. The method comprising administering to the subject fusion protein disclosed herein. In some implementations, the subject is administered the fusion protein orally. In other implementations, the subject is administered the fusion protein by intravenous, intraarterial, intramuscular, intraperitoneal, subcutaneous, intradermal, intraosseous, intracardiac, intraarticular, intracavernous, periarticular, or intracranial injection. In certain implementations, the subject is administered the fusion protein via injection of a composition comprising an adenovirus having a nucleic acid sequence encoding the fusion protein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1H depict leveraging LRRC10 modulation of Ca_{V}1.2 channels to develop a genetically encoded enhancer of L-type currents. FIG. 1A provides exemplary whole-cell recordings that show robust baseline Ca_{V}1.2 currents in HEK 293 cells. Top, exemplar traces show Ca_{V}1.2 current evoked by 15 ms depolarizations to a family of test pulse potentials. Bottom, aggregate data for current density-voltage (I-V) relationship. FIG. 1B shows that co-expression of full-length LRRC10 markedly increases Ca_{V}1.2 current density. The left part of FIG. 1C depicts a cartoon illustrates a FRET pair with Cerulean-tagged LRRC10 or subsegments serving as FRET donor while Venus-tagged holo-Ca_{V}1.2 serving as FRET acceptor. The right part of FIG. 1C depicts a flow-cytometry based live cell FRET 2-hybrid assay demonstrating that Ca_{V}1.2 interacts robustly with full length LRRC10. Further dissecting LRRC10 revealed that the CT domain containing the Lrr domains suffice for binding to Ca_{V}1.2, while NT domain exhibits poor binding. Statistical analysis: One-way ANOVA followed by Tukey's multiple comparison test. **** p<0.0001. Although LRRC10 CT suffices for strong interaction with Ca_{V}1.2, this domain is insufficient for functional channel modulation (FIG. 1D). LRRC10 NT also fails to alter Ca_{V}1.2 currents (FIG. 1E). The right part of FIG. 1F depicts a cartoon showing the structure-guided design of GeeC (genetically encoded enhancer of Ca_{V}1.2/1.3 channels) by fusing the LRRC10 NT with nb.F3 that targets Ca_{V}1.2. Co-expression of nb.F3 minimally alters peak Ca_{V}1.2 current density (FIG. 1G). GeeC markedly enhances peak L-type current density in HEK 293 cells (FIG. 1H). For FIGS. 1A, 1B, 1D, 1G, and 1H: n=number of cells; Error bars show mean and s.e.m.

FIGS. 2A-2F demonstrate that GeeC selectively enhances Ca_{V}1.2/1.3 currents. Co-expression of GeeC increases Ca_{V}1.3 currents in HEK 293 cells compared to nb.F3 (FIG. 2A). Inset shows exemplar Ca_{V}1.2 recordings in the presence of nb.F3 or GeeC. GeeC minimally perturbs peak current density of Ca_{V}1.4 channels (FIG. 2B). GeeC fails to alter peak current density of Ca_{V}2.1 (FIG. 2C), Ca_{V}2.2 (FIG. 2D), and Ca_{V}2.3 (FIG. 2E) channels. For FIGS. 2A-2E: Error bars show mean and s.e.m. FIG. 2F depicts bar graphs comparing peak current density for various Ca_{V}1/2 channels in the presence of nb.F3 or GeeC. Error bars show mean and s.e.m. Statistical analysis: Kolmogorov-Smirnov normality test followed by Mann-Whitney test (α1C, α1A and α1F) and unpaired Student's t-test (α1B, α1D and α1E). * p<0.05. *** p<0.001. n=14 and 24 (±GeeC, Ca_{V}1.2); n=7 and 9 (±GeeC, Ca_{V}1.3); n=6 and 9 (±GeeC, CaV1.4); n=10 and 11 (±GeeC, Ca_{V}2.1); n=9 and 7 (±GeeC, Ca_{V}2.2); and n=13 and 10 (±GeeC, Ca_{V}2.3).

FIGS. 3A-3I demonstrate that GeeC upregulates Ca_{V}1.2 function by enhancing channel openings. Exemplar cell-attached single channel recordings of Ca_{V}1.2 reconstituted in HEK 293 cells (FIG. 3A). Co-expression of GeeC increases channel openings while nb.F3 yields minimal change (FIGS. 3B and 3C). For FIGS. 3A-3C, slanted gray curve represents unitary conductance. Each downward deflection indicates channel openings. Ensemble average P_{O}-voltage relationship for Ca_{V}1.2 shows that GeeC increases peak P_{O} compared to control conditions or in the presence of nb.F3. n=6 (control), n=6 (nb.F3) and n=7 (GeeC) from 3 independent transfections (FIG. 3D). The schematic of FIG. 3E shows dual labeling approach to quantify surface expression of Ca_{V}1.2. The pore-forming α1C subunit is engineered to contain a bungarotoxin binding site in DII S5-S6 loop and a YFP at the carboxy-terminus. Surface expression can be quantified by labeling with bungarotoxin conjugated to Alexa Fluor 647 (SA647), while total expression is determined by measuring YFP fluorescence (SYFP). Flow cytometric analysis shows baseline levels of Ca_{V}1.2 surface membrane trafficking in the presence of β2b subunits (FIG. 3F). Coexpression of GeeC (FIG. 3G) or nb.F3 (FIG. 3H) minimally perturbs surface expression of Ca_{V}1.2. Population data confirms minimal change in surface-membrane expression of Ca_{V}1.2 in the presence of either GeeC or nb.F3 (FIG. 3I). Each dot, geometric mean of Alexa Fluor 647 staining from an individual experiment. Bars show mean and s.e.m.

FIGS. 4A-4E show GeeC enhances endogenous Ca_{V}1.2 in mouse-derived cardiomyocytes. Brightfield (top) and epifluorescence (bottom) images show a cardiomyocyte expressing GeeC, with mCherry bicistronically expressed (FIG. 4A). Exemplar traces of endogenous L-type currents in 2-day cultured cardiomyocytes evoked in response to 300 ms voltage steps to various potentials (FIG. 4B, top). Population data show baseline peak current density. (FIG. 4B, bottom). n=11 from 3 mice Adenoviral expression of GFP minimally perturbs L-type current density (FIG. 4C). n=8 from 3 mice. GeeC markedly upregulates endogenous L-type channels (FIG. 4D). n=11 from 3 mice. Nb.F3 by itself failed to appreciably perturb peak current density (FIG. 4E). n=8 from 2 mice. Error bars represent mean and s.e.m.

FIGS. 5A-5I show that GeeC boosts excitation-transcription coupling in neurons. Confocal images show increased nuclear accumulation of pCREB (Alexa Fluor 647) following cell depolarization with extracellular solution containing 40 mM K⁺ (FIG. 5A). White dashed circles mark the nuclear region for fluorescence quantification based on DAPI staining. Expression of GFP has minimal effect on nuclear pCREB levels following depolarization (FIG. 5B). Expression of GeeC increases nuclear pCREB staining following depolarization (FIG. 5C). Population data compares nuclear pCREB signal following depolarization (FIG. 5D). Bars show mean±s.e.m. Statistical analysis: Kruskal-Wallis test followed by Dunn's multiple comparisons test. **** p<0.0001. n=133 (control, resting); 122 (control; with depolarization); 95 (GFP); 110 (GeeC) cells from 6 independent cultures. To probe functionality of GeeC in vivo, AAV9 encoding either GFP or GeeC/mCherry was stereotactically injected into the mouse hippocampus. Confocal image of a coronal slice of mouse hippocampi at 10× magnification shows robust expression of GeeC (mCherry) or GFP control AAV9 (FIG. 5E, top), and pCREB signal (FIG. 5E, bottom). pCREB staining is qualitatively higher in the right hemisphere with GeeC expression. Higher magnification (40×) confocal images of mouse dorsal hippocampal neurons showing GFP (FIG. 5F, left) or mCherry (FIG. 5F, right) and corresponding pCREB staining. Representative coronal slice of medial prefrontal cortex at 10× magnification shows either GeeC or GFP expression (FIG. 5G, left), and corresponding pCREB staining (FIG. 5G, right). FIG. 5H depicts higher magnification images of mice prefrontal cortex neurons. The top portion shows GFP or GeeC expression, while the bottom portion shows pCREB. Population data confirms enhanced pCREB staining in GeeC-expressing prefrontal cortex neurons when compared to GFP-infected controls (FIG. 5I). Statistical analysis: Mann-Whitney test. **** p<0.0001. Bars show mean±s.e.m.

FIGS. 6A-6I show that GeeC boosts E-T coupling in Rett Syndrome (RTT) neurons. Exemplar confocal images show robust increase in nuclear pCREB staining following mild membrane depolarization in wildtype hESC-derived neurons (FIG. 6A). Left, baseline; Right, following 40 mM K+ stimulation. Scale bar, 5 μm. Expression of GFP in wildtype neurons minimally perturbs recruitment of nuclear pCREB (FIG. 6B). Scale bar, 5 μm. Accumulation of pCREB in the nucleus following membrane depolarization is markedly reduced in RTT-like neurons (FIG. 6C). Scale bar, 5 μm. L-type channel upregulation by GeeC enhances pCREB signaling in RTT neurons (FIG. 6D). Scale bar, 5 μm. Bar graph summarizes population data showing increase in pCREB following membrane depolarization in both wild-type and RTT neurons (FIG. 6E). Statistical analysis, Kruskal-Wallis test followed by Dunn's multiple comparisons test. **** p<0.0001. n=73 (uninfected, wildtype), n=63 (GFP, wildtype), 128 (GFP, RTT), and 96 (GeeC, RTT) cells from 3 independent cultures. Confocal images show differences in overall morphology of wild-type (FIG. 6F) versus RTT neurons in the presence of GFP (FIG. 6G) or GeeC (FIG. 6H). Blue, DAPI; Yellow, Tuj 1; Red, mCherry; Green, GFP. Scale bar, 10 μm. (I) Bar graph quantifies changes in soma size in both wild-type and RTT neurons. Each bar and error, mean±s.e.m. Statistical analysis, Kruskal-Wallis test followed by Dunn's multiple comparisons test. * p=0.0114; **** p<0.0001. n=44 (uninfected, wildtype), n=20 (GFP, wildtype), 155 (GFP, RTT), and 120 (GeeC, RTT) cells from 3 independent cultures.

### DETAILED DESCRIPTION OF THE INVENTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices, and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components. As used herein, the term “nb.F3” refers to a llama nanobody first described in Morgenstern et al., “A potent voltage-gated calcium channel inhibitor engineered from a nanobody targeted to auxiliary Ca_{V}β subunits,” Elife, 2019, 8: e49253. The nanobody was isolated from immunization with Ca_{V}β subunits (β1-4).

A L-type Ca²⁺ channel (LTCC) is a complex of multiple independent protein subunits, which are proteins that facilitate the function of the voltage-gated channel. Among these, leucine-rich repeat-containing protein 10 (LRRC10) is a cardiac-specific member of the superfamily. In zebrafish, LRRC10 is essential for cardiac function, including cardiac development. Specifically, LRRC10 knockdown leads to reduced cardiomyocyte population, and cardiac dysmorphism. In mice, deletion of LRRC10 results in reduced cardiac function immediately after birth, progressing to dilated cardiomyopathy in adulthood. In addition, these mice exhibit reduced contractility in response to pressure overload induced by transverse aortic constriction. Furthermore, human LRRC10 variants are associated with pediatric dilated cardiomyopathy and cardiac arrhythmias. Recently, LRRC10 has also been found to be an essential element for heart regeneration in both zebrafish and in mammals; however, how LRRC10 mediates this effect is not fully understood. LRRC10 is known to associate with multiple proteins, including the α-subunit of the Ca_{V}1.2 channel, α-actinin, and actin.

Similar to other leucine-rich repeat (Lrr) containing proteins, LRRC10 is composed of seven stereotypic Lrr domains with signature sequences LxxLxLxxNxL (SEQ ID NO. 1) or LxxLxLxxNxxL (SEQ ID NO. 2) flanked by short N-terminal (NT) and C-terminal (CT) regions. The Lrr domains are thought to enable binding to disparate targets, allowing this diverse family of proteins to serve a wide range of cellular functions. LRRC10's high affinity interaction with Ca_{V}1.2 is mediated by the Lrr-domains, similar to LRRC10 interaction with other targets. Lrr domains are thought to mediate protein-protein interactions and may promiscuously support association with diverse targets. Critically, functional Ca_{V}1.2 upregulation also requires the NT of LRRC10, which serves as a low affinity effector domain. Unlike the Lrr domains which share considerable homology with other Lrrc family of proteins, the NT region is unique to LRRC10 and is highly conserved across species. This suggests that while other members of the Lrrc family of proteins may also interact with the Ca_{V}1.2 channel, it is unlikely that these proteins would serve as Ca_{V}1.2/1.3 agonists. It is possible that LRRC10 binds to the carboxy-terminal domain of Ca_{V}1 channels, which serves as a nexus for interacting with other regulatory proteins, including calmodulin and stac proteins, both of which upregulate Ca_{V}1 P_{O}.

The current understanding of the physiology of LRCCs and their pathophysiological consequences are derived largely from either constitutive or cell-type specific knockout of Ca_{V}1.2 and/or Ca_{V}1.3 in mice. By comparison, there are limited strategies available for targeted upregulation Ca_{V}1.2/1.3 function. This limitation hampers an in-depth understanding of the biological mechanisms.

From an experimental perspective, development of next-generation customizable Ca_{V}1 actuators is highly desirable to illuminate complex physiology. Pharmacologically, Ca_{V}1 blockers including dihydropyridines and phenylalkylamines, are used clinically for various indications (e.g. hypertension, migraine and arrhythmias). Genetically encoded inhibitors of Ca_{V} channels have also been engineered by leveraging channel modulation by Rem/Gem/Kir family of G-proteins, or by targeted ubiquitination of channel complexes. Conversely, activation of Ca_{V}1 channels via non-tissue specific pharmacological Ca_{V}1 agonists can be deleterious. Administration of Ca_{V}1 agonists Bay K 8644 and FPL 64176 result in severe motor abnormalities, seizure, and self-harming behavior in rodents. Therefore, alternative strategies for targeted or tissue specific upregulation of Ca_{V}1 function are needed to prevent off-target toxicities. Beyond the pore-forming subunit that is targeted by conventional pharmacology, Ca_{V} channels associate with a large group of regulatory proteins that fine-tune channel dynamics. Leveraging an intracellular regulatory protein may provide an alternative strategy to engineer a genetically encodable Ca_{V} enhancer.

Precise manipulation of Ca_{V}1.2/Ca_{V}1.3 in distinct physiological settings is crucial for delineating their biological functions and for broadening their utility as a therapeutic target. GeeC was engineered as a genetically encoded approach to boost Ca_{V}1.2/1.3 function with enhanced specificity in various physiological settings. To do so, a nanobody that binds the CaVβ subunit was functionalized by attaching a low affinity effector domain from LRRC10. The present disclosure utilizes a recently identified Ca_{V}1.2 modulator LRRC10, which enhances Ca_{V}1.2 current in heterologous systems and ventricular cardiomyocyte. LRRC10 has been implicated in cardiomyocyte maturation, regeneration, and response to increased afterload. The present invention discloses a minimal effector domain within LRRC10 that upregulates Ca_{V}1.2, albeit with a low affinity such that it poorly modulates channel function by itself. Conjugation of this minimal effector domain to a targeting agent that binds the Ca_{V} complex with a high affinity, termed genetically encoded enhancer of Ca²⁺ channels (GeeC), yielded a Ca_{V}1.2 and Ca_{V}1.3 specific actuator, which significantly increases the channel open probability (P_{O}). GeeC targets the Ca_{V}β subunit to modulate channel function. As there are differences in cytosolic domains of Ca_{V}1.2 versus Ca_{V}1.3, in certain embodiments, nanobodies are engineered to specifically target a Ca_{V} channel. In certain embodiments, GeeC is a constitutive enhancer of Ca_{V}1. In some embodiments, the NT domain of LRRC10 is recruited to the targeting domain (i.e. nanobody) through chemically-induced or optogenetic dimerizers.

In depth analysis revealed that GeeC selectively modulates Ca_{V}1.2 and Ca_{V}1.3 channels by upregulating channel P_{O}, while minimally perturbing closely related Ca_{V} channels. In cardiomyocytes, GeeC expression results in increased Ca_{V}1.2 current density, confirming its functionality in modulating native Ca²⁺ channels. In cultured hippocampal neurons, GeeC expression increased pCREB staining, suggesting increased excitation-transcription coupling. Furthermore, GeeC delivery into mouse hippocampus and medial prefrontal cortex increased pCREB in neurons, pointing to enhanced E-T coupling in vivo. Thus, GeeC provides a new avenue to upregulate Ca_{V}1.2/1.3 function in various physiological settings.

As shown in the Examples, GeeC can be administered to cardiomyocytes and neurons to modulate endogenous Ca_{V}1.2 and Ca_{V}1.3 currents, and to tune downstream physiological functions. Of note, as Lrr repeats often interact promiscuously with protein targets, and this chimeric approach provides a potential platform for targeted upregulation of CaV channels in distinct physiological settings, avoiding potential off-target effects.

Accordingly, a fusion protein that modulates Ca_{V}1.2 and/or Ca_{V}1.3 channel activity and enhances L-type currents is disclosed. The fusion protein, also referred to herein as GeeC, leverages both LRRC10 and selective targeting agent that target the LTCC complex (for example a nanobody) to increase Ca_{V}1.2 and/or Ca_{V}1.3 channel activity and to enhances L-type currents in a cell. The fusion protein comprises a polypeptide derived from LRRC10 and a targeting agent, wherein the targeting agent is conjugated to the polypeptide, and the targeting agent is selective for a β subunit of a LTCC. In some aspects, the targeting agent has a greater binding affinity to the β subunit of the LTCC than the polypeptide. In some embodiments, the LTCC is Ca_{V}1.2 or Ca_{V}1.3. In some embodiments where the targeting agent is a nanobody, the nanobody is functionalized through attachment of fluorescent reporters or enzymes that alter channel post-translational modifications to tune channel localization.

The polypeptide enhances channel activity of Ca_{V}1.2 and/or Ca_{V}1.3. Thus, in some aspects, the polypeptide comprises the amino-terminus (NT) domain of LRRC10 or a portion thereof. In some aspects, the amino acid sequence of the polypeptide comprises the sequence set forth in SEQ ID NO. 4. In some embodiments, the polypeptide comprises first 53 amino acids of LRRC10. In certain implementations, the polypeptide comprises less than 60 amino acids. In particular embodiments, the polypeptide is derived from a human LRRC10. Accordingly, the NT domain of LRRC10 has the sequence set forth in SEQ ID NO. 4.

In some embodiments of the fusion protein, the targeting agent is a nanobody. For example, the targeting agent is nb.F3. Thus in certain embodiments, the amino acid sequence of the fusion protein is set forth in SEQ ID NO. 5. In certain implementations, the nucleic acid sequence encoding the fusion protein is set forth in SEQ ID NO. 6.

In other aspects, a peptide conjugate comprising a polypeptide derived from LRRC10 conjugated to a targeting agent selective for a β subunit of a LTCC is disclosed. As with the fusion peptide, the peptide conjugate increase Ca_{V}1.2 and/or Ca_{V}1.3 channel activity and to enhances L-type currents in a cell. In some embodiments, the polypeptide comprises the NT domain of LRRC10 or a portion thereof. In some aspects, the amino acid sequence of the polypeptide comprises the sequence set forth in SEQ ID NO. 4. In some embodiments, the polypeptide comprises first 53 amino acids of LRRC10. In certain implementations, the polypeptide comprises less than 60 amino acids. In particular embodiments, the polypeptide is derived from a human LRRC10. Accordingly, the NT domain of LRRC10 has the sequence set forth in SEQ ID NO. 4. In some aspects, the targeting agent has a greater binding affinity to the β subunit of the LTCC (for example, Ca_{V}1.2 or Ca_{V}1.3) than the polypeptide.

Also described herein are a method of producing the above-described fusion protein. The method comprises synthesizing a nucleic acid sequence that encodes the fusion protein, wherein the nucleic acid sequence comprises a first nucleic acid sequence encoding the NT domain of LRRC10 or a fragment thereof, a second nucleic acid sequence encoding the targeting agent (for example, a nucleic acid sequence encoding a nanobody), and a third nucleic acid sequence encoding a linker sequence that links polypeptide and the nanobody. In some aspects, the method further comprises introducing the nucleic acid sequence that encodes the fusion protein into an adenovirus vector and introducing the nucleic acid sequence that encodes the fusion protein into an adenovirus using the adenovirus vector.

In a particular embodiment, GeeC was synthesized as a DNA fragment by isolating the first 53 amino acids of human LRRC10 and linking them to nb.F3-P2A peptide through a GSGRSGSG sequence (SEQ ID NO. 3), flanked by NheI/EcoRI sites (Twist Bioscience). The gene fragment was ligated using T4 DNA ligase (Thermo Fisher) into a PiggyBac CMV mammalian expression vector that encoded a CFP sequence downstream of a P2A peptide. Ligates were then transformed into either XL10-Gold Ultracompetent Cells (Agilent) or DH5a Competent Cells (Thermo Fisher), plated and cultured in selective LB broth or Circlegrow Medium (MP Biomedicals). DNA was extracted and purified from cultures using either QIAprep Spin Miniprep Kit (Qiagen) or GeneJET PCR Purification Kit (Thermo Fisher). Sanger (Eton Bioscience Inc. and Genewiz from Azenta Life Sciences) and Nanopore sequencing (SNPsaurus, LLC) were used to verify plasmids. FRET plasmids were generated by PCR amplification of full-length 360 human LRRC10, LRRC10 NT, and LRRC10 LRR1-CT using primers flanked by restriction sites NheI or BglII/SalI. Sequences were then ligated into a pcDNA3 plasmid coding for a downstream GSG-Cerulean (Cer) sequence immediately after restriction site.

Several pharmacological Ca_{V}1 blockers, including dihydropyridines and verapamil, are routinely used clinically to downregulate Ca_{V}1 activity for various indications. Although Ca_{V}1.2/1.3 agonists have been postulated as potential treatment for multiple diseases including bladder dysfunction, these approaches pose inherent challenges. Previous studies that used Bay K 8644, a dihydropyridine, in rodents reported increased coronary vascular resistance, self-biting behavior, and motor abnormalities including twisting and stretching movements, limb extension, back arching, spasticity, and ataxia. A key advantage of GeeC is that it is genetically encoded and may be delivered to specific cell types using emerging viral gene delivery approaches. As shown in the Examples, low affinity functional domains from channel interacting proteins or other targets could also be used as payloads for tuning specific aspects of channel function, such as specific aspects of Ca_{V} channel function or specific aspects of Na_{V} channel function. Recent studies have identified a number of short peptides that tune specific properties of Ca_{V}, and Na_{V} channels. Thus, further disclosed herein are pharmaceutical compositions comprising the fusion protein, the peptide conjugate, or the nucleic acid sequence encoding the fusion protein. In some aspects, the pharmaceutical composition comprises a pharmaceutically acceptable additive, for example, a diluent, a carrier, an excipient, a stabilizer, a coating, or capsule shells.

In some embodiments where the pharmaceutical composition comprises the fusion protein, the composition further comprises an adenovirus. In such embodiments, the adenovirus has been transfected with an adenovirus vector, and the adenovirus vector comprises the nucleic acid sequence encoding the fusion protein.

In some embodiments where the pharmaceutical composition comprises the nucleic acid sequence encoding the fusion protein, the composition further comprises an adenovirus vector comprising the nucleic acid sequence encoding the fusion protein.

As the examples show that LRRC10 co-expression markedly upregulates LTCC currents, a method of enhancing L-type currents in a cell is disclosed. The method comprises administering to the cell the fusion protein or peptide conjugate disclosed herein. Thus, use of the fusion protein or peptide conjugate to enhance L-type in a cell is also disclosed. In some embodiments, the fusion protein or peptide conjugate for enhancing L-type currents comprises a polypeptide comprising the NT domain of LRRC10 (or a portion thereof) and a targeting agent selective for a β subunit of LTCC, wherein the targeting agent is conjugated to the polypeptide. In some implementations, the fusion protein is administered to the cell via transducing the cell with an adenovirus. In such implementations, the adenovirus comprises a nucleic acid sequence encoding the fusion protein. In some implementations, the adenovirus has been transfected with an adenovirus vector comprising a transgene that encodes the fusion protein.

In mouse cardiomyocytes, transduction of GeeC via adenovirus enhances endogenous L-type currents when compared to controls or cardiomyocytes infected with unconjugated nanobodies alone. Additionally, in neurons, co-expression of GeeC enhanced excitation-transcription coupling. Thus, in certain implementations, methods of enhancing L-type currents in neurons and muscle cells (for example cardiomyocytes) are disclosed.

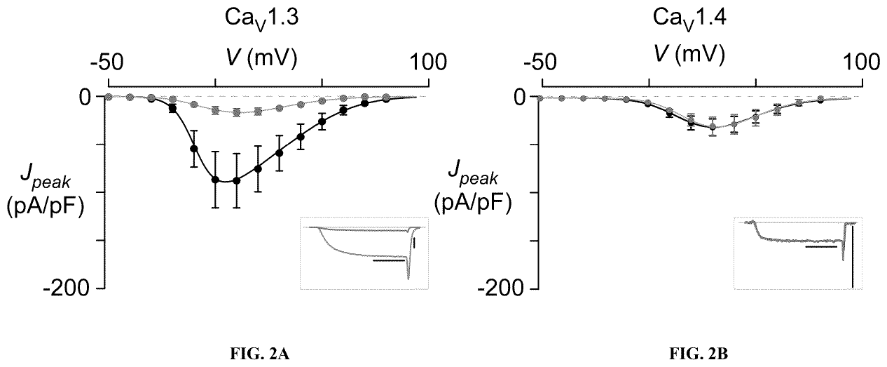

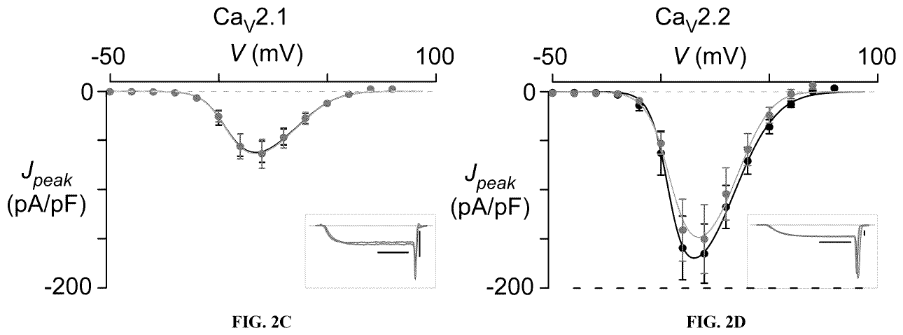

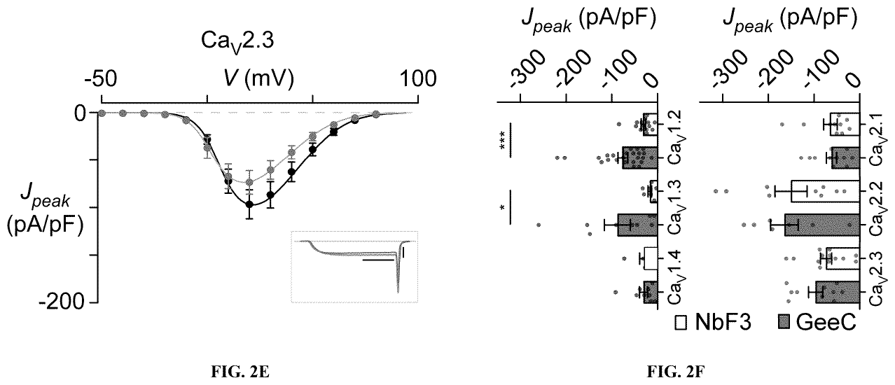

Having confirmed the ability of GeeC to enhance Ca_{V}1.2 currents, selectivity of GeeC in tuning various members of the Ca_{V} channel superfamily was characterized. Similar to Ca_{V}1.2, that GeeC yielded a marked increase in Ca_{V}1.3 currents compared to co-expression of nb.F3 (FIGS. 2A and 2F). By contrast, the closely related Ca_{V}1.4 channels were entirely unaffected (FIGS. 2B and 2F). Furthermore, GeeC had minimal effect on Ca_{V}2 channels (FIGS. 2C-2F). These findings demonstrate that GeeC is selective in upregulating Ca_{V}1.2 and Ca_{V}1.3 channels. As such, a method of upregulating Ca_{V}1.2 and/or Ca_{V}1.3 current in a cell is disclosed. The method comprises administering to the cell the fusion protein or peptide conjugate disclosed herein. Thus, use of the fusion protein or peptide conjugate to upregulate Ca_{V}1.2 and/or Ca_{V}1.3 current in a cell is also disclosed. In some embodiments, the fusion protein or peptide conjugate comprises a polypeptide for upregulating Ca_{V}1.2 and/or Ca_{V}1.3 current comprises the NT domain of LRRC10 (or a portion thereof) and a targeting agent selective for a β subunit of LTCC, wherein the targeting agent is conjugated to the polypeptide. In some implementations, the fusion protein is administered to the cell via transducing the cell with an adenovirus. In such implementations, the adenovirus comprises a nucleic acid sequence encoding the fusion protein. In some implementations, the adenovirus has been transfected with an adenovirus vector comprising a transgene that encodes the fusion protein. In certain implementations of the method, Ca_{V}1.2 and/or Ca_{V}1.3 current in neurons and muscle cells (for example cardiomyocytes) is upregulated.

As shown in the Examples, boosting L-type channel function can reverse pathophysiological signaling in a neuronal model of Rett syndrome. Specifically, RTT neurons show a baseline reduction in E-T coupling, which could be reversed by expression of GeeC. Thus, further disclosed herein is a method of reducing neurological symptoms of Rett Syndrome (RTT) and the use of the fusion protein or peptide conjugate disclosed herein for treating RTT. RTT is an X-linked neurodevelopmental disorder associated with ASD, hypotonia, microcephaly, stereotyped limb movements, and an initial phase of normal development, followed by regression of acquired developmental milestones. RTT is caused by loss-of-function mutations in methyl CpG-binding protein 2 (MECP2), a transcriptional regulator. As MeCP2 is a downstream target of L-type Ca²⁺ channels and as decreased gene expression has been reported in RTT, altered E-T coupling in RTT neurons was determined, and whether modulation of L-type Ca²⁺ channel activity by GeeC could perturb this process in the pathophysiological setting.

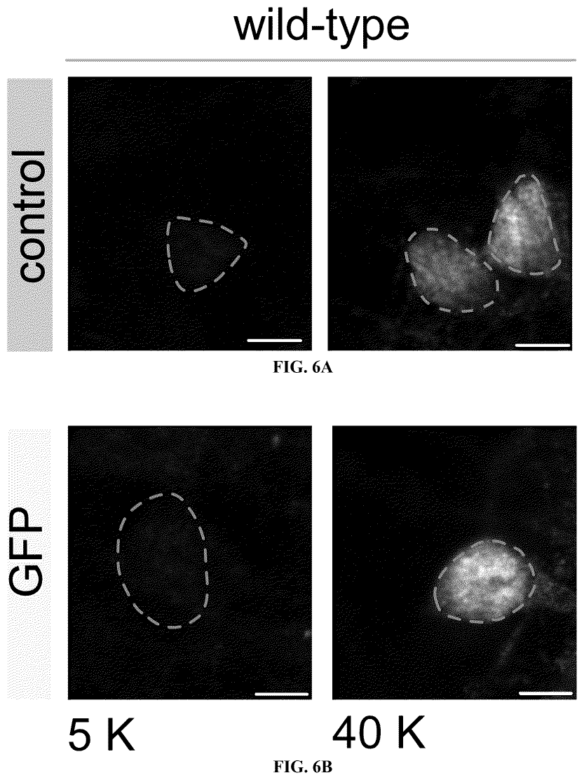

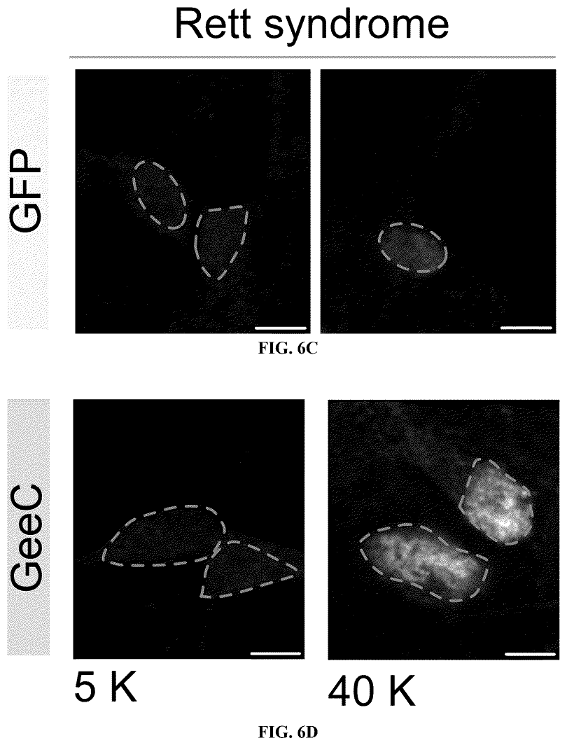

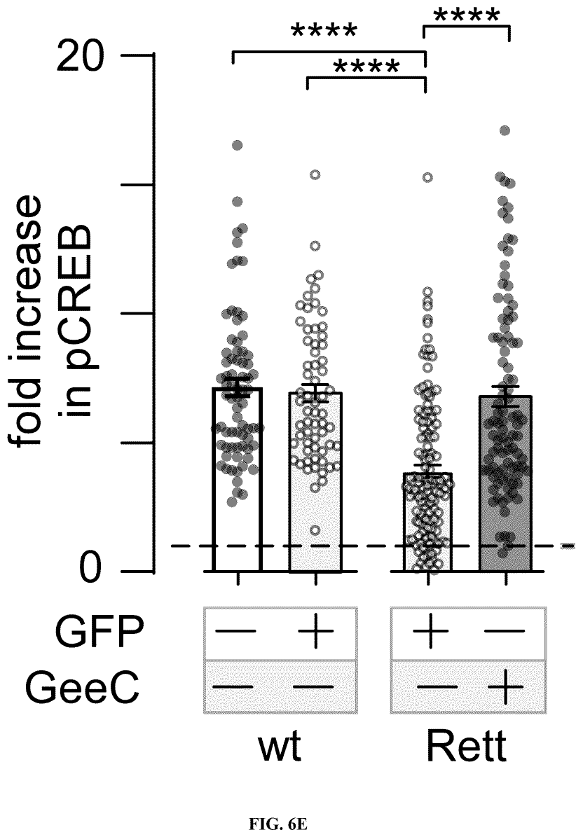

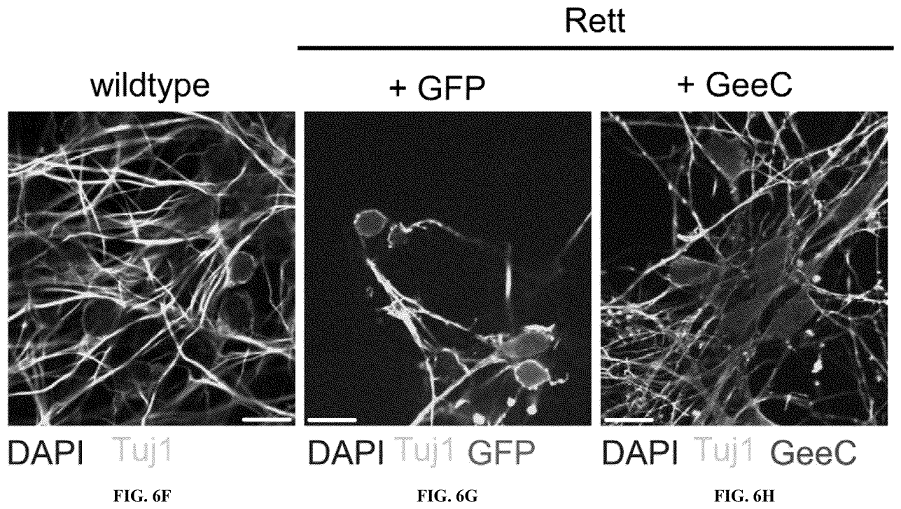

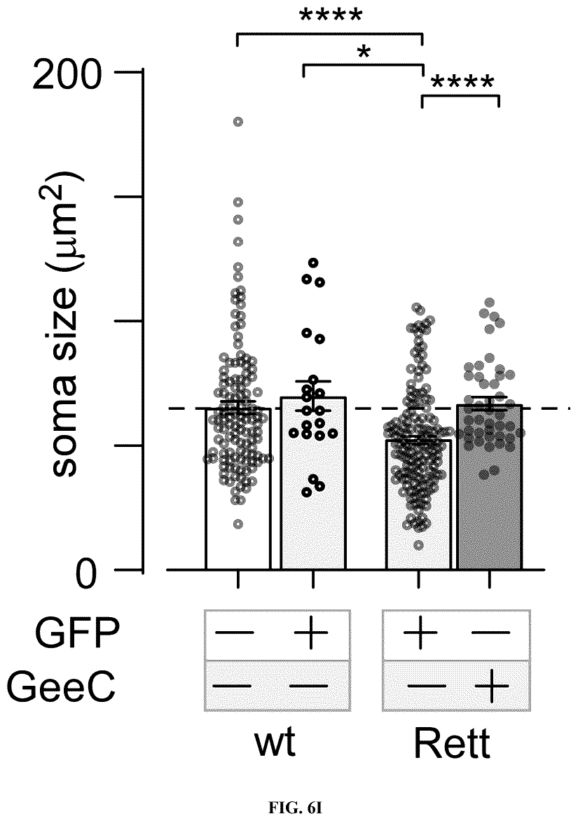

Compared to control or GFP-expressing wild-type neurons (FIGS. 6A, 6B, 6E), GFP-expressing RTT neurons exhibited markedly reduced nuclear pCREB (FIGS. 6C and 6E). GeeC overexpression reversed this reduction in pCREB to near wild-type levels (FIGS. 6D and 6E). Expression of GeeC increased soma size, reversing it to near-wild type soma size (FIGS. 6H and 6I). Thus, GeeC can boost E-T coupling in RTT neurons, which can compensate for signaling deficits in Rett Syndrome. Accordingly, the method reducing neurological symptoms of RTT in a subject diagnosed with RTT comprises administering to the subject the fusion protein or peptide conjugate disclosed herein. For example, the administered fusion protein or peptide conjugate comprises a polypeptide comprising the NT domain of LRRC10 (or a portion thereof) and a targeting agent selective for a β subunit of LTCC, wherein the targeting agent is conjugated to the polypeptide. In some embodiments, the fusion protein or peptide conjugate is administered as a pharmaceutical composition disclosed herein.

In some implementations, the subject is administered the fusion protein or peptide conjugate orally. In other implementations, the subject is administered the fusion protein or peptide conjugate by intravenous, intraarterial, intramuscular, intraperitoneal, subcutaneous, intradermal, intraosseous, intracardiac, intraarticular, intracavernous, periarticular, or intracranial injection. In such implementations, the fusion protein is administered by injecting the subject with an adenovirus, the adenovirus comprising a nucleic acid sequence encoding the fusion protein.

From a molecular engineering perspective, GeeC is a versatile and generalizable platform to devise Ca_{V}1.2 and Ca_{V}1.3 actuators with desired properties. As there are differences in cytosolic domains of Ca_{V}1.2 versus Ca_{V}1.3, it may be possible to engineer nanoparticles that target the channels directly. At present, GeeC is a constitutive enhancer of Ca_{V}1. However, it may be feasible to engineer GeeC such that LRRC10 NT is recruited to the targeting domain (i.e. nanobody) through chemically-induced or optogenetic dimerizers. More broadly, nanobodies have emerged as a powerful platform to probe and manipulate ion channel function. Nanobodies have been functionalized through attachment of fluorescent reporters or enzymes that alter channel post-translational modifications to tune channel localization. The presented disclosure suggests low affinity functional domains from channel interacting proteins or other targets could also be used as payloads for tuning specific aspects of channel function. This strategy may be advantageous, as recent studies have identified a number of short peptides that tune specific properties of Ca_{V}, and Na_{V} channels. In all, GeeC enables targeted upregulation of Ca_{V}1.2 and Ca_{V}1.3 channels in diverse physiological settings, thereby opening new avenues to development of future therapeutics, and providing a convenient strategy to systematically dissect the function of these channels in a cell-type dependent manner.

The invention is further described by the following numbered paragraphs:
1. A fusion protein comprising:
   a polypeptide derived from leucine-rich repeat-containing protein 10 (LRRC10); and
   a targeting agent,
   wherein:
   the targeting agent is conjugated to the polypeptide;
   the targeting agent is selective for a β subunit of a L-type Ca²⁺ channel (LTCC); and
   the polypeptide modulates channel activity of Ca_{V}1.2 and/or Ca_{V}1.3.
2. The fusion protein of paragraph 1, wherein the polypeptide enhances channel activity of Ca_{V}1.2 and/or Ca_{V}1.3.
3 The fusion protein of paragraph 1 or 2, wherein the polypeptide comprises the amino-terminus (NT domain) of LRRC10 or a portion thereof.
4. The fusion protein of any one of paragraphs 1-3, wherein the polypeptide comprises first 53 amino acids of LRRC10.
5. The fusion protein of any one of paragraphs 1-4, wherein the polypeptide has an amino acid sequence comprising less than 60 amino acids.
6. The fusion protein of any one of paragraphs 1-5, wherein the targeting agent has a greater binding affinity to the β subunit of LTCC than the polypeptide.
7. The fusion protein of any one of paragraphs 1-6, wherein the LTCC is Ca_{V}1.2 or Ca_{V}1.3.
8. The fusion protein of any one of paragraphs 1-7, wherein the targeting agent is a nanobody.
9. The fusion protein of paragraph 8, wherein the nanobody is nb.F3.
10. The fusion protein of any one of paragraphs 1-9, wherein the polypeptide is derived from human LRRC10.
11. The fusion protein of any one of paragraphs 1-10, wherein the amino acid sequence of the fusion protein is set forth in SEQ ID NO. 5.
12. The fusion protein of any one of paragraphs 1-10, wherein the nucleic acid sequence encoding the fusion protein is set forth in SEQ ID NO. 6.
13. A pharmaceutical composition comprising:
   a polypeptide derived from leucine-rich repeat-containing protein 10 (LRRC10); and
   a targeting agent,
   wherein:
   the targeting agent is conjugated to the polypeptide;
   the targeting agent is selective for a β subunit of a L-type Ca²⁺ channel (LTCC); and
   the polypeptide modulates channel activity of Ca_{V}1.2 and/or Ca_{V}1.3.
14. A pharmaceutical composition comprising a nucleic acid sequence encoding the fusion protein of any one of paragraphs 1-12.
15. The pharmaceutical composition of paragraph 14 comprising an adenovirus vector, wherein the adenovirus vector comprises the nucleic acid sequence encoding the fusion protein.
16. The pharmaceutical composition of paragraph 14, further comprising an adenovirus, wherein the adenovirus has been transfected with an adenovirus vector, and the adenovirus vector comprises the nucleic acid sequence encoding the fusion protein.
17. The pharmaceutical composition of any one of paragraphs 14-16, wherein the nucleic acid sequence encoding the fusion protein is set forth in SEQ ID NO. 6.
18. The pharmaceutical composition of any one of paragraph 13-17, further comprising a pharmaceutically acceptable additive.
19. A method of upregulating Ca_{V}1.2 and/or Ca_{V}1.3 current in a cell, the method comprising:
   administering to the cell a fusion protein comprising:
   a polypeptide derived from leucine-rich repeat-containing protein 10 (LRRC10); and
   a targeting agent,
   wherein:
   the targeting agent is conjugated to the polypeptide;
   the targeting agent is selective for a β subunit of a L-type Ca²⁺ channel (LTCC); and
   the polypeptide comprises the amino-terminus (NT domain) of LRRC10 or a portion thereof.
20. A method of enhancing L-type currents in a cell, the method comprising administering to the cell a fusion protein comprising:
a polypeptide derived from leucine-rich repeat-containing protein 10 (LRRC10); and
a targeting agent,
wherein:
the targeting agent is conjugated to the polypeptide;
the targeting agent is selective for a β subunit of a L-type Ca²⁺ channel (LTCC); and
the polypeptide comprises the amino-terminus (NT domain) of LRRC10 or a portion thereof.
21. The method of paragraph 19 or 20, wherein the cell is a neuron or a muscle cell.
22. The method of paragraph 21, wherein the muscle cell is a cardiomyocyte.
23. The method of any one of paragraph 19-22, wherein the fusion protein is administered to the cell via transducing the cell with an adenovirus, the adenovirus comprising a nucleic acid sequence encoding the fusion protein.
24. A method of reducing neurological symptoms in a subject diagnosed with Rett Syndrome (RTT), the method comprising:
   administering to the subject a fusion protein comprising:
   a polypeptide derived from leucine-rich repeat-containing protein 10 (LRRC10); and
   a targeting agent,
   wherein:
   the targeting agent is conjugated to the polypeptide;
   the targeting agent is selective for a β subunit of a L-type Ca²⁺ channel (LTCC); and
   the polypeptide comprises the amino-terminus (NT domain) of LRRC10 or a portion thereof.
25. The method of paragraph 24, wherein the subject is administered the fusion protein orally.
26. The method of paragraph 24, wherein the subject is administered the fusion protein by intravenous, intraarterial, intramuscular, intraperitoneal, subcutaneous, intradermal, intraosseous, intracardiac, intraarticular, intracavernous, periarticular, or intracranial injection.
27. The method of paragraph 26, wherein the fusion protein is administered by injecting the subject with an adenovirus, the adenovirus comprising a nucleic acid sequence encoding the fusion protein.

### EXAMPLES

### Example 1. Leveraging LRRC10 Modulation to Engineer a Ca_{V}1.2 Actuator

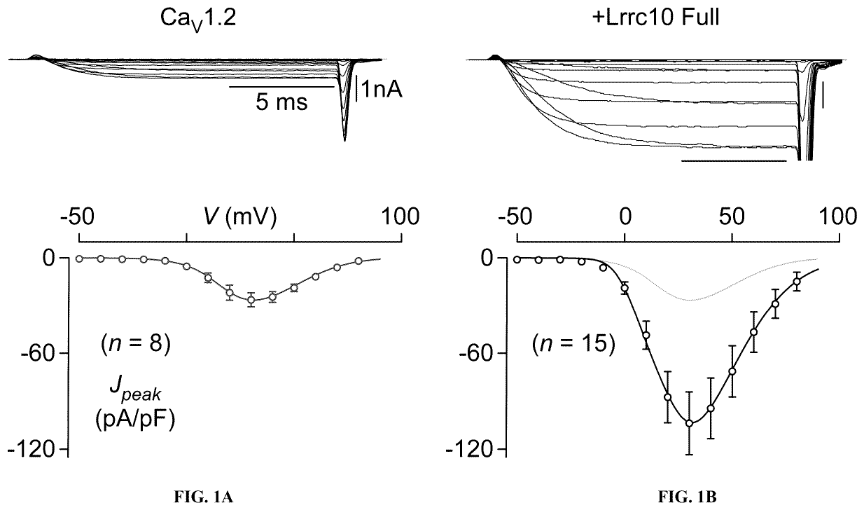

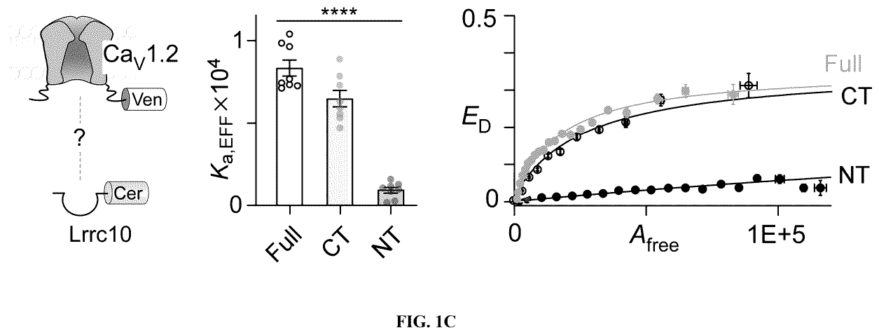

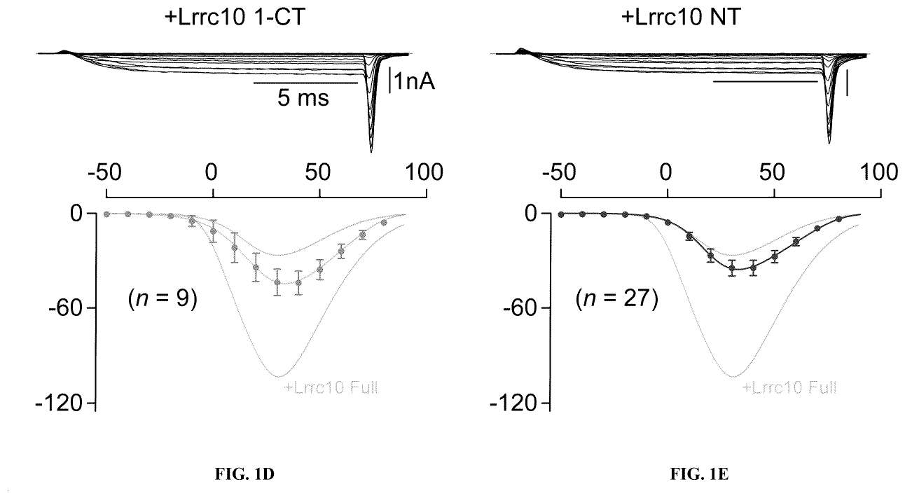

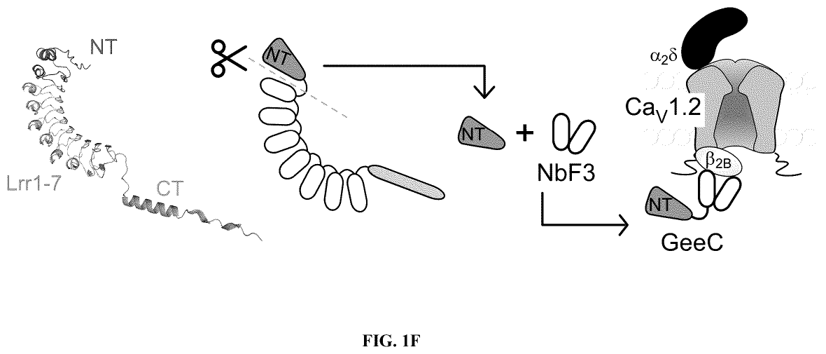

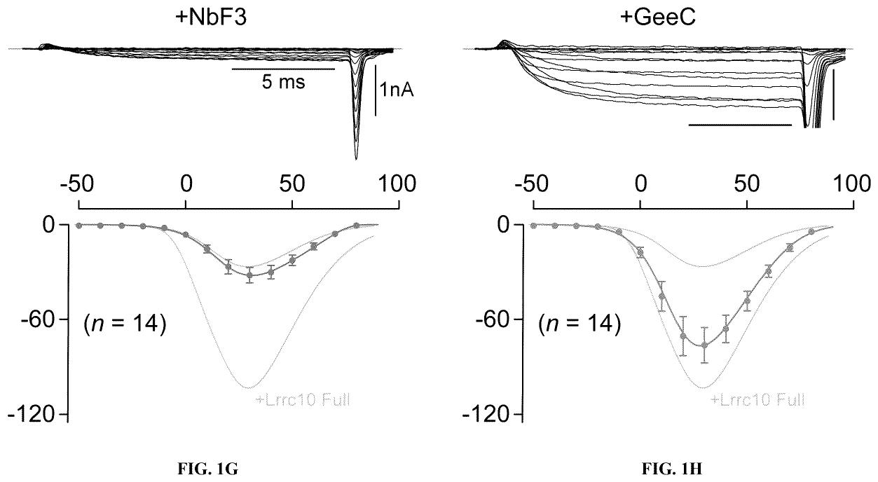

To quantify the effect of LRRC10 on L-type Ca²⁺ currents, peak current density was measured from whole cell recordings of Ca_{V}1.2, co-transfected with auxiliary subunits α2δ1 and β2b in HEK 293 cells. When compared to cells expressing only Ca_{V}1.2 canonical subunits (FIG. 1A), co-expression of LRRC10 yielded a marked increase in peak current density (FIG. 1B), consistent with previous reports. Given this striking increase in current density, key segments within LRRC10 responsible for binding and modulating Ca_{V} channels were identified. Similar to other leucine-rich repeat (Lrr) containing proteins, LRRC10 is composed of seven stereotypic Lrr domains with signature sequences LxxLxLxxNxL (SEQ ID NO. 1) or LxxLxLxxNxxL (SEQ ID NO. 2) flanked by short N-terminal (NT) and C-terminal (CT) regions. Traditionally, the Lrr domains are thought to enable binding to disparate targets, allowing this diverse family of proteins to serve a wide range of cellular functions. A flow cytometry-based FRET 2-hybrid assay was used to probe interaction of LRRC10 segments with holo-Ca_{V}1.2 in live cells (FIG. 1C). Specifically, LRRC10 was bisected into (1) an N-terminal (NT) domain, and (2) a C-terminal (CT) segment that included the Lrr domains, and attached cerulean fluorescent protein, the FRET donor, to each peptide. Venus fluorescent protein, the FRET acceptor, was attached to the carboxy terminus of Ca_{V}1.2. Indeed, full length LRRC10 displayed robust FRET binding with holo-Ca_{V}1.2 (FIG. 1C). Furthermore, the LRRC10 CT segment also revealed strong binding to Ca_{V}1.2 with a relative association constant (K_{a,EFF}) that is nearly as robust as full-length LRRC10 (FIG. 1C). By comparison, the isolated LRRC10 NT domain showed weak to no binding (FIG. 1C). These results suggest that the LRRC10 CT containing the Lrr domains is critical for robust Ca_{V}1.2 interaction.

Thus affirmed, whether LRRC10 CT was sufficient for functional modulation of Ca_{V}1.2 was assessed. Surprisingly, co-expression of LRRC10 CT only minimally changed Ca_{V}1.2 peak current density, despite its ability to interact with full length Ca_{V}1.2 (FIG. 1D). Furthermore, co-expression of the LRRC10 NT domain also revealed little to no change in peak current density, likely reflecting its weak affinity for Ca_{V}1.2 (FIG. 1E). Taken together, these results point to two distinct possibilities: (1) the overall tertiary structure of LRRC10 may be essential for functional Ca_{V}1.2 modulation, such that any alteration in its structure abrogates its function; or (2) LRRC10 may be composed of two functionally distinct domains: the CT segment containing the Lrr domains may be responsible for high affinity binding to Ca_{V}1.2, while the NT segment may be a low affinity effector domain responsible for driving Ca²⁺ current upregulation.

If LRRC10 CT serves as a binding domain for Ca_{V}1.2, then replacing this region with a peptide or a domain that interacts with Ca_{V}1.2 while preserving LRRC10 NT would maintain functional modulation. In this regard, recent studies have reported a nanobody that binds to the Ca_{V}β-subunit, nb.F3, with a high affinity (~10 nM), but that is functionally inert. As such, a chimeric protein was engineered by fusing LRRC10 NT region with nb.F3 and probed its effect on tuning Ca_{V}1.2 currents (FIG. 1F). Indeed, co-expression of nb.F3 with Ca_{V}1.2 yielded no change in peak current density, consistent with the nanobody being functionally silent (FIG. 1G). However, co-expression of the chimeric LRRC10-NT fused to nb.F3 resulted in a nearly 3-fold increase in peak current density (FIG. 1H). Given its ability to upregulate Ca_{V}1.2 currents, this chimeric protein was named “genetically encoded enhancer of Ca²⁺ currents” or GeeC. Overall, these findings are consistent with the bi-modular architecture of LRRC10, whereby the NT serves as a low affinity effector domain to increase currents, while the CT serves as a binding domain that is responsible for interacting with Ca_{V}1.2. Of note, as Lrr repeats often interact promiscuously with protein targets, this chimeric approach provides a potential platform for targeted upregulation of Ca_{V} channels in distinct physiological settings, avoiding potential off-target effects.

### Example 2. GeeC Selectively Upregulates Ca_{V}1.2/1.3 Channels

Having confirmed the ability of GeeC to enhance Ca_{V}1.2 currents, selectivity of GeeC in tuning various members of the Ca_{V} channel superfamily was determined. All members of the Ca_{V}1/Ca_{V}2 family are known to interact with the Ca_{V}β subunit targeted by GeeC. As such, it is possible that any of these channels may be modulated by GeeC. By comparison, Ca_{V}3 channels do not require the Ca_{V}β subunit for its function and are unlikely to be affected by GeeC. Accordingly, changes in peak current density of other Ca_{V} channels upon co-expression with GeeC was systematically quantified. Similar to Ca_{V}1.2, GeeC yielded a marked increase in Ca_{V}1.3 currents compared to co-expression of nb.F3 (FIGS. 2A and 2F). By contrast, the closely related Ca_{V}1.4 channels were entirely unaffected (FIGS. 2B and 2F). Furthermore, GeeC had minimal effect on Ca_{V}2 channels (FIGS. 2C-2F). These findings demonstrate that GeeC is selective in upregulating Ca_{V}1.2/1.3 channels.

### Example 3. Mechanism of Ca_{V}1.2/1.3 Upregulation by GeeC

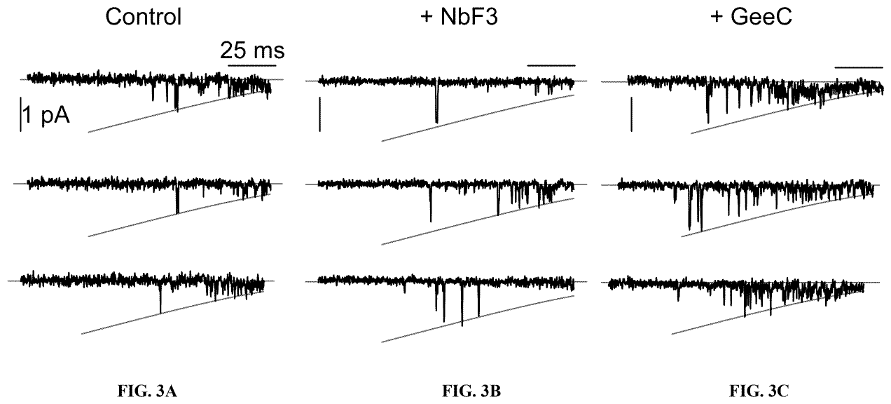

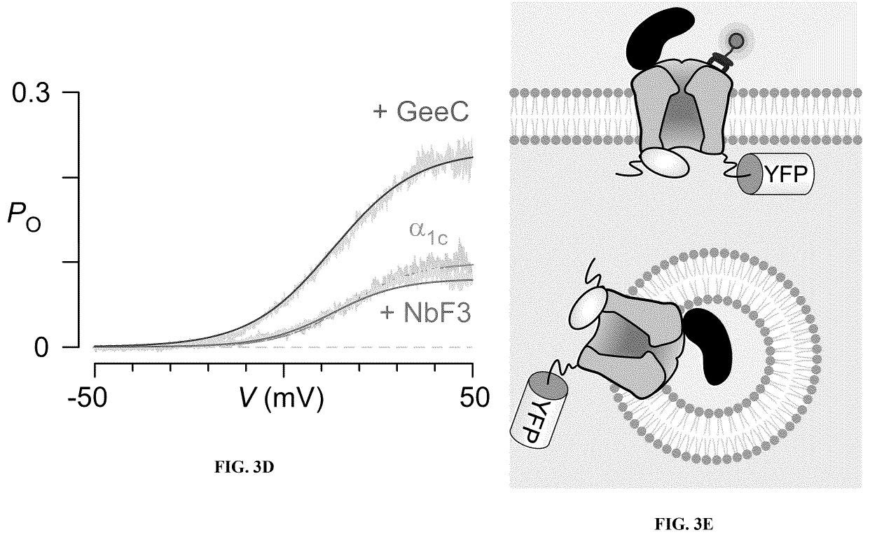

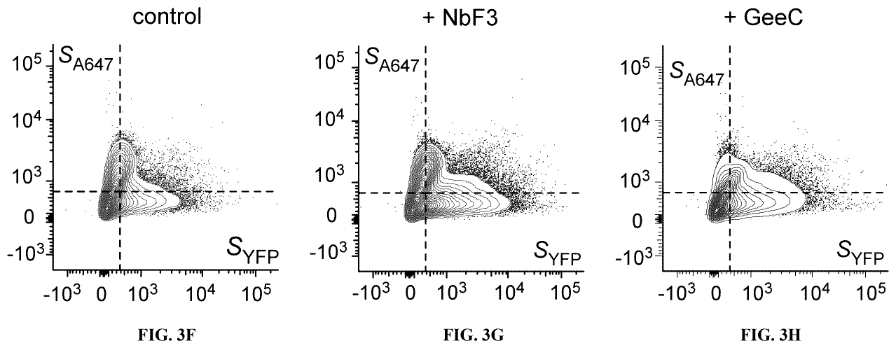

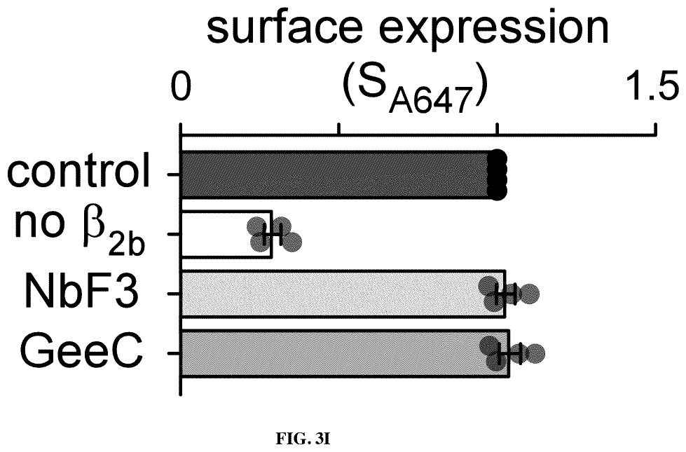

Mechanistically, GeeC may upregulate Ca_{V}1.2/1.3 whole cell current by enhancing one of three fundamental elementary channel properties: (1) the open probability (P_{O}), i.e. the likelihood that channel is open at any given voltage; (2) the unitary current (i), which describes the current through a single open channel, or (3) the total number of channels (N) at the surface membrane. To distinguish between these possibilities, low-noise, cell-attached, single-channel recordings of Ca_{V}1.2 heterologously expressed were taken along with α2δ1 and β2B in HEK 293 cells (FIGS. 3A-3D). These recordings permit direct measurement of both PO and i while preserving the intracellular milieu. Here, Ba²⁺ was used as charge carrier to avoid confounding effects of Ca²⁺-dependent inactivation. Stochastic channel openings were evoked at near steady-state P_{O} at each voltage using a slow voltage-ramp. FIG. 3A shows elementary channel openings for Ca_{V}1.2 as downward deflections to the unitary current level at each voltage (gray slanted curve). Co-expression of GeeC (FIG. 3B) but not nb.F3 (FIG. 3C) increased number of openings. The steady-state P_{O}-voltage relationship was then estimated by averaging 80-150 stochastic records to obtain a mean current that is divided into the open level and averaged over multiple patches. GeeC increased the maximal P_{O} compared to control conditions or with nb.F3 alone (FIG. 3D).

To determine whether GeeC alters surface-membrane trafficking of Ca_{V}1.2, a dual labeling approach was used (FIG. 3E). Specifically, the pore-forming α-subunit of Ca_{V}1.2 was engineered to contain tandem bungarotoxin (BTX) binding sites on a loop exposed to the extracellular surface, and a yellow fluorescent protein (YFP) on its carboxy-terminus. With this assay, the total number of channels in single cells can be measured as the fluorescence intensity of YFP, while the surface-membrane channels can be selectively labeled by incubating cells with BTX conjugated to Alexa Fluor 647. Robust baseline surface expression was observed for Ca_{V}1.2 when co-expressed with β2b as reported by normalized Alexa Fluor 647 signal (FIGS. 3F and 3I). As in previous studies, Ca_{V}1.2 surface expression is markedly reduced in the absence of β2b subunit (FIG. 3I). By comparison, co-expression of GeeC or nb.F3 minimally altered surface-membrane trafficking of Ca_{V}1.2 channels (FIGS. 3G-3I). Taken together, these results suggest that GeeC enhances Ca_{V}1.2 function by boosting channel openings.

### Example 4. GeeC Enhances L-Type Current in Cardiomyocytes

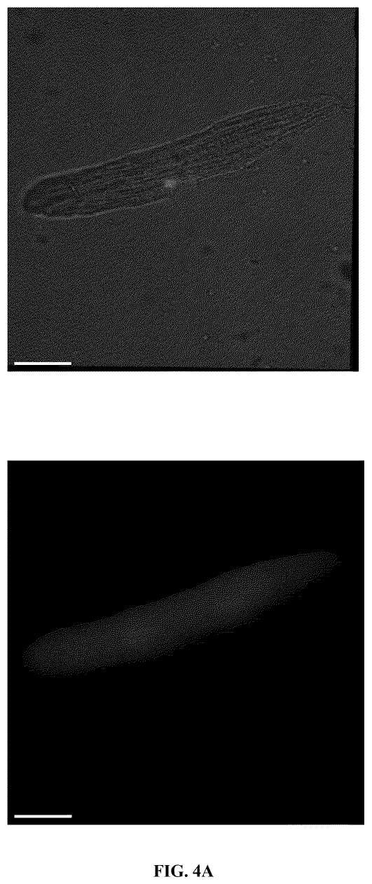

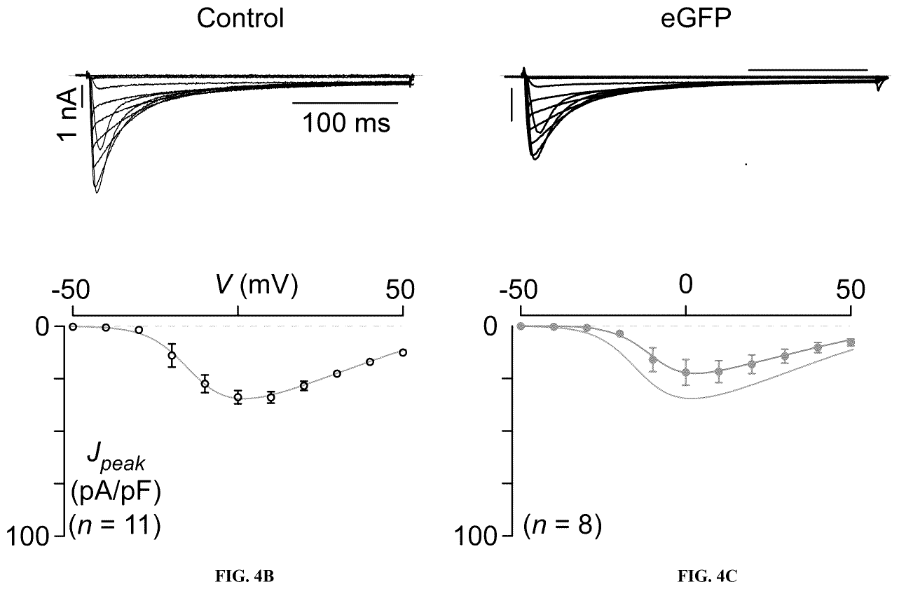

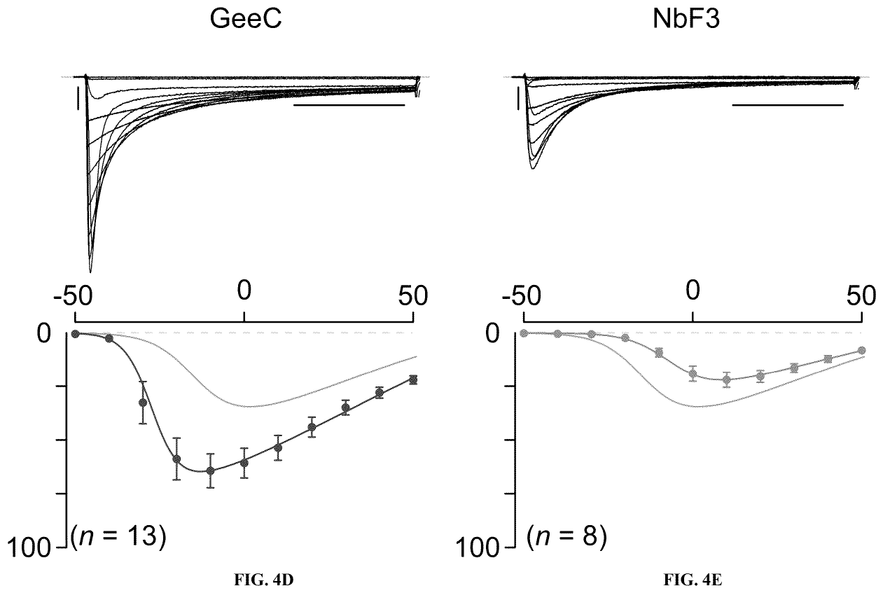

Having established the functionality of GeeC, whether GeeC can upregulate native L-type current in cardiomyocytes was determined. Ventricular cardiomyocytes isolated from adult mice, which primarily express Ca_{V}1.2 channels, were cultured, and then adenovirus was used to transduce GeeC along with a bicistronically-expressed mCherry as fluorescent reporter into these cells (FIG. 4A). Whole cell current recordings show baseline Ca²⁺ currents in these cells evoked in response to a family of voltage-step depolarizations (FIG. 4B). Adenoviral transduction of green fluorescent protein (GFP) revealed a minimal change in peak current density (FIG. 4C). By contrast, GeeC expression yielded a marked increase in Ca²⁺ current density (FIG. 4D). Expression of nb.F3 alone failed to upregulate the Ca²⁺ current density, with peak current density matching levels observed with GFP expression (FIG. 4E). These findings demonstrate the ability of GeeC to enhance native Ca_{V}1.2 channels in cardiomyocytes.

### Example 5. GeeC Boosts Excitation-Transcription Coupling in Neurons In Vitro and In Vivo

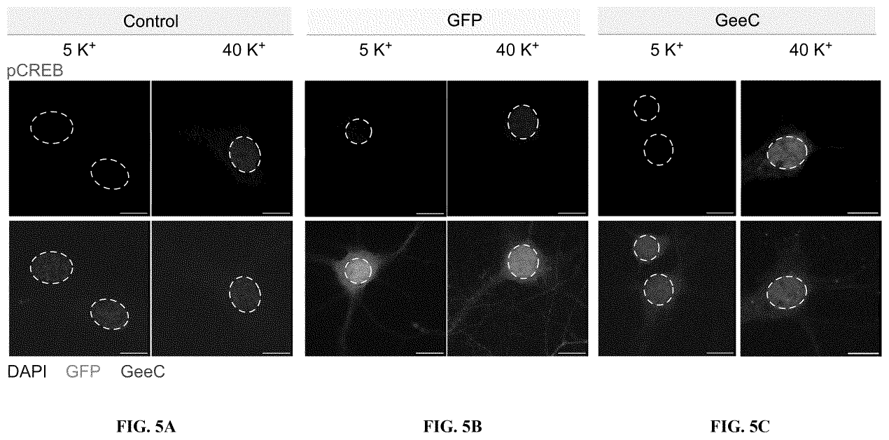

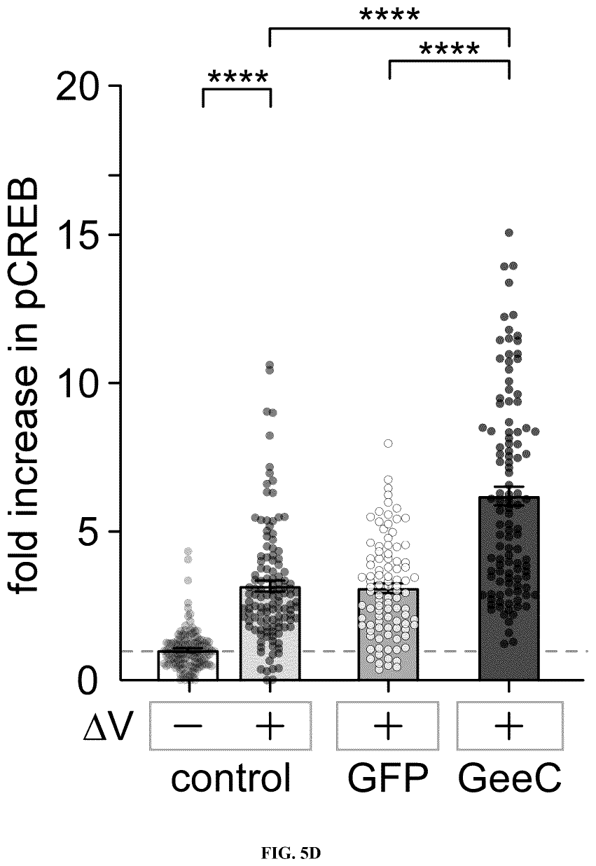

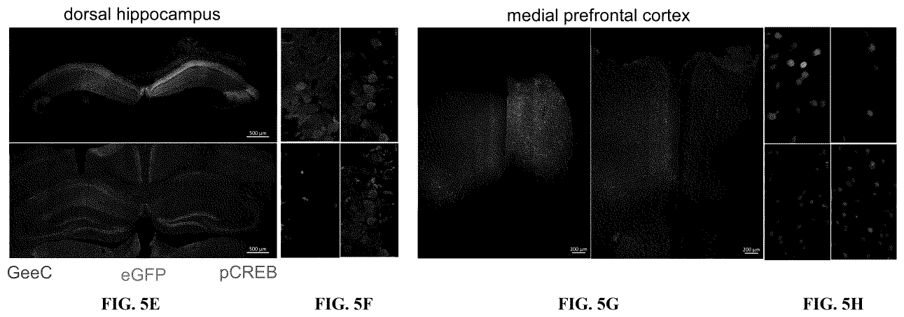

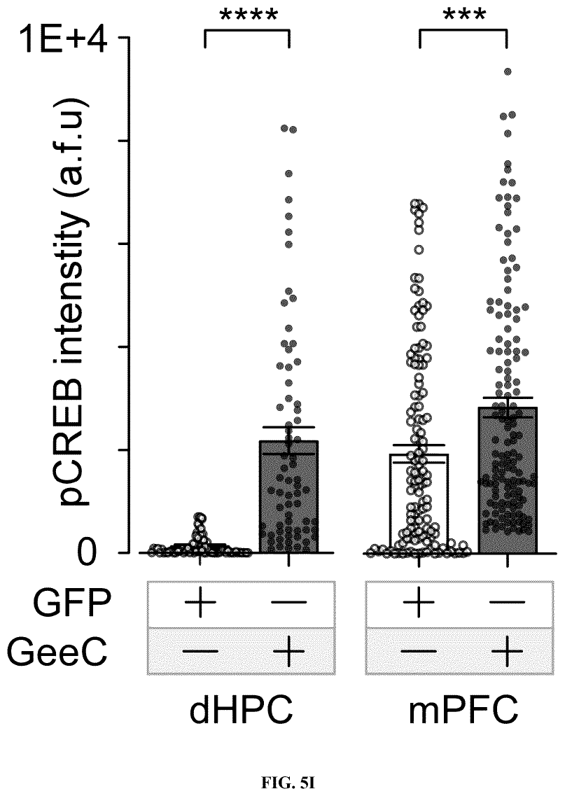

In neurons, Ca_{V}1.2/1.3 channels play an essential role in transducing membrane excitation to changes in gene expression, a process known as excitation-transcription (E-T) coupling. This process is critical for driving neurodevelopmental changes and in modulating synaptic plasticity. Briefly, Ca²⁺ entry through NMDA and Ca_{V}1.2/1.3 channels, as well as voltage-dependent conformation changes in the latter, leads to autophosphorylation of Ca²⁺/calmodulin-dependent protein kinase II (CaMKII). This, in turn, results in activation of downstream CaMKII pathways and an increase in immediate early genes via phosphorylation of nuclear cyclic AMP-response element binding protein (pCREB), a transcription factor. Either GeeC or GFP was transduced into cultured rat hippocampal neurons to assess whether. upregulation of Ca_{V}1.2/1.3 by GeeC may boost excitation-transcription coupling (FIGS. 5A-5I). As in previous studies, neurons were depolarized with 40 mM extracellular potassium (K+) that preferentially evokes signaling through Ca_{V}1 channels. The strength of excitation-transcription coupling was then measured by immunostaining of nuclear pCREB. Without depolarization, minimal pCREB was observed in the nucleus (FIGS. 5A and 5D). Following depolarization, a significant increase in pCREB staining in the nucleus was observed (FIGS. 5A and 5D). Adenoviral expression of GFP resulted in minimal changes in depolarization-induced pCREB in the nucleus (FIGS. 5B and 5D). By comparison, expression of GeeC resulted in a nearly 2-fold increase in nuclear pCREB staining compared to uninfected and GFP controls (FIGS. 5C and 5D). These findings reveal the ability of GeeC to augment excitation-transcription coupling in cultured neurons, likely reflecting an enhancement in Ca_{V}1.2/1.3 function.

Having confirmed its effect in cultured neurons, whether GeeC can boost excitation-transcription coupling in the mouse brain was assessed. Using stereotaxic surgical techniques, 12-14 weeks old mice were unilaterally injected with either AAV9-GeeC (right hemisphere) or AAV9-GFP (left hemisphere)—as negative control—in the dorsal hippocampus (FIG. 5E) and the medial prefrontal cortex (FIG. 5G). Following 2.5 weeks of recovery, robust expression of both GeeC (mCherry marker) and GFP were observed in the respective hemispheres in the dorsal hippocampus (FIG. 5E) and medial prefrontal cortex (FIG. 5G) brain sections. pCREB was probed via immunostaining. In the dorsal hippocampus, AAV9-GeeC injected hippocampal neurons had higher levels of nuclear pCREB compared to AAV9-GFP control injected neurons (FIGS. 5F and 5I). Similar effects were also observed in mPFC neurons, with GeeC-expressing neurons demonstrating a higher pCREB staining compared to GFP-infected control (FIGS. 5H and 5I). In all, these findings illustrate the exquisite capability of GeeC to modulate excitation-transcription coupling in neurons both in vitro and in vivo.

### Example 6. GeeC Enhances Excitation—Transcription Coupling in Rett Syndrome

Rett syndrome (RTT) is an X-linked neurodevelopmental disorder associated with ASD, hypotonia, microcephaly, stereotyped limb movements, and an initial phase of normal development, followed by regression of acquired developmental milestones. RTT is caused by loss-of-function mutations in methyl CpG-binding protein 2 (MECP2), a transcriptional regulator. As MeCP2 is a downstream target of L-type Ca²⁺ channels and as decreased gene expression has been reported in RTT, whether RTT neurons have altered E-T coupling and whether modulation of L-type Ca²⁺ channel activity by GeeC could perturb this process in the pathophysiological setting were probed. To do so, a previously established RTT-like human embryonic stem cell (hESC) line that was genetically engineered from a wild-type female hESC line was differentiated into neurons in vitro (RTT neurons herein) was used. These RTT neurons been shown to recapitulate key cellular alterations associated with RTT, including reduced soma size compared to neurons from healthy donors and reduced induction of various intermediate early genes following extracellular K⁺ mediated depolarization. To test if enhancing L-type Ca²⁺ channels with GeeC can rescue these features, the neuronal precursor cells derived from this RTT-like hESC were infected with either AAV9-GFP or GeeC, and then characterized the infected neurons. First, baseline changes in E-T coupling in RTT neurons compared to wild-type neurons was probed by measuring nuclear pCREB following depolarization. Compared to control or GFP-expressing wild-type neurons (FIGS. 6A, 6B, and 6E), GFP-expressing RTT neurons exhibited markedly reduced nuclear pCREB (FIGS. 6C and 6E). GeeC overexpression reversed this reduction in pCREB to near wild-type levels (FIGS. 6D and 6E). Additionally, whether GeeC expression could rescue the reduced soma size was tested. Consistent with previous studies, Tuj1 staining used to measure soma size revealed ~25% reduction in GFP-expressing RTT neurons compared to wildtype neurons (FIGS. 6F, 6G, and 6I). Expression of GeeC increased soma size, reversing it to near-wild type soma size (FIGS. 6H and 6I). Taken together, these findings demonstrate that GeeC can boost E-T coupling in RTT neurons and potentially compensate for signaling deficits in Rett Syndrome.

These findings are consistent with previously-reported reduction in activity-induced expression of intermediate early genes in these cells, and reduced CREB signaling in similar cell lines and mice. Upregulation of Ca_{V}1.2/1.3 function by GeeC also reversed deficits in soma size, a cellular phenotype of RTT neurons. Previous work has shown that upregulating CREB signaling, downstream of Ca_{V}1 activation, in RTT neurons can enhance neurite outgrowth growth and dendritic complexity. It is currently unknown whether there are deficits in Ca_{V}1.2/1.3 function in RTT neurons. Gene-expression analysis has shown reduced expression of both Ca_{V}β1 and Ca_{V}β3 subunits in MECP2 mice and increased expression of both subunits in MECP2-overexpressing models. Nevertheless, it is possible that upregulation of Ca_{V}1.2/1.3 by GeeC may be compensatory for other signaling changes. More broadly, in the brain, inducible expression of GeeC in distinct neuron populations or specific neural circuits may allow us to delineate the complex contribution of Ca_{V}1 function to elicit a variety of behavioral responses in animal models, or to dissect the importance of Ca_{V}1 in formation of neural circuits in the developing brain through inducible expression in early developmental stages. In the heart, GeeC could be utilized to selectively target Ca_{V}1.2 in cardiomyocytes, potentially increasing E-C coupling, inotropy and cardiac output. This may be advantageous to delineate the discrete roles for augmented Ca²⁺ entry versus phosphorylation in heart remodeling and cardiac disease, where prolonged adrenergic stimulation plays a pathophysiological role.

### Example 7. Materials and Methods

a. Molecular Biology

GeeC was synthesized as a DNA fragment by isolating the first 53 amino acids of human LRRC10 and linking them to nb.F3-P2A peptide through a GSGRSGSG (SEQ ID NO. 3) sequence, flanked by NheI/EcoRI sites (Twist Bioscience). The gene fragment was ligated using T4 DNA ligase (Thermo Fisher) into a PiggyBac CMV mammalian expression vector that encoded a CFP sequence downstream of a P2A peptide. Ligates were then transformed into either XL10-Gold Ultracompetent Cells (Agilent) or DH5a Competent Cells (Thermo Fisher), plated and cultured in selective LB broth or Circlegrow Medium (MP Biomedicals). DNA was extracted and purified from cultures using either QIAprep Spin Miniprep Kit (Qiagen) or GeneJET PCR Purification Kit (Thermo Fisher). Sanger (Eton Bioscience Inc. and Genewiz from Azenta Life Sciences) and Nanopore sequencing (SNPsaurus, LLC) were used to verify plasmids. FRET plasmids were generated by PCR amplification of full-length human LRRC10, LRRC10 NT, and LRRC10 LRR1-CT using primers flanked by restriction sites NheI or BglII/SalI. Sequences were then ligated into a pcDNA3 plasmid coding for a downstream GSG-Cerulean (Cer) sequence immediately after restriction site.

b. Adenovirus and AAV Generation

Plasmids, adenovirus (pAV[Exp]-mCherry-CMV>[GEECC2.0] and pAV[Exp]-CMV>EGFP) and AAV9 (pAAV [Exp]-CMV>[GEECC2.0]:BGHpA-hPGK>mCherry:WPRE and pAAV [Exp]-CMV>EGFP:WPRE) were constructed and packaged by VectorBuilder. GeeC adenovirus vector ID is VB220916-1205wzd (sequence set forth in SEQ ID NO. 7). GeeC AAV9 vector ID is VB230125-1077nkn (sequence set forth in SEQ ID NO. 8). GFP adenovirus vector ID is VB010000-9299hac (sequence set forth in SEQ ID 9). GFP AAV9 vector ID is VB010000-9394npt (sequence set forth in SEQ ID NO. 10). CFP-P2A-nb.F3 adenovirus was used as in previous studies (Morgenstern et al., “A potent voltage-gated calcium channel inhibitor engineered from a nanobody targeted to auxiliary Ca(V) beta subunits.” Elife 8, (2019)).

c. HEK 293 Cell Culture and Transfection

HEK 293 cells (American Type Culture Collection, catalog no. CRL1573) were cultured on glass coverslips in 60-mm dishes and transfected using a calcium phosphate method. For whole-cell electrophysiology, 4 μg of cDNA encoding rabbit α1C subunit (NM_001136522) along with 4 μg of human β2b subunit (AF285239.2) and 4 μg of rat brain α2δ1 (NM_012919.2) were applied. In experiments that required so, co-transfection with 4 μg of either GeeC or nb.F3 were perfumed. To enhance expression, cDNA for simian virus 40 T antigen (0.5 mg) was co-transfected. Electrophysiology recordings were done at room temperature 24 hours after transfection. For single-channel electrophysiology, 1 μg of α1C, β2b and α2δ1, along 1 μg of either GeeC or nb.F3 were applied. For total and surface Ca²⁺ channel flow-cytometric assay, 3 μg of engineered α1C and β2b subunits along with either nb.F3 or GeeC were transfected. To enhance expression, cDNA for simian virus 40 T antigen (0.5 mg) was co-transfected.

d. Cardiomyocyte Isolation, Culture and Infection

Mice ventricular myocytes were isolated from 8- to 12-week-old non-transgenic (C57BL/6) mice by enzymatic digestion using a Langendorff perfusion apparatus. Both male and female mice were used to obtain isolated cardiomyocytes, and experimental outcomes were unaffected by sex. Cardiomyocytes were resuspended in perfusion solution with 5% fetal bovine serum (General Electric). CaCl2 was added over 30 minutes to a final concentration of 1 mM. Cells were plated on glass coverslips precoated with laminin (Corning). Plating medium contained minimal essential medium (MEM) with Earle's salts and L-glutamine, 5% fetal bovine serum, 1% penicillin-streptomycin and 10 mM 2,3-butanedione monoxime (BDM). Cardiomyocytes were allowed to precipitate and adhere for 2 h before changing to ‘maintenance’ medium, which contained MEM with Earle's salts, L-glutamine, 1% penicillin-streptomycin, bovine serum albumin (0.5 mg ml-1), 10 mM BDM, 1% insulin-transferrin-selenium, 5 mM creatine, 5 mM taurine, 2 mM L-carnitine and 25 UM blebbistatin. For deployment of GeeC, nb.F3, or GFP, 1-2 μl of adenovirus were resuspended in the culture medium at least 2 hours after cell plating. Maintenance medium was replaced after 12-20 h to reduce potential adenoviral toxicity. Viral transduction efficacy was assessed 24-48 h post-infection. Only non-contracting, rod-shaped cardiomyocytes with clear striations were used for electrophysiology.

e. Isolated Rat Hippocampal Neuron Culture and Infection

Culturing kits for rat hippocampi at embryonic age 18 were procured from Transetyx Tissue by BrainBits (Tranetyx Inc). Neurons were isolated from hippocampi using papain in Ca²⁺-free medium (2 mg/mL) and gentle mechanical dissociation. Cells were plated in 12-well plates containing glass cover slips precoated with poly-D-lysine (50 μg/mL) at a final concentration of 16,000 cells/cm². Neurons were cultured initially in NbActiv1 culture medium (BrainBits) and switched to Neurobasal/B27/Glutamax ‘maintenance’ medium (Gibco) 96 h after plating. Medium changes were done subsequently every 72 h. GeeC and GFP were adenovirally-transduced on culture day 10 by resuspending 0.5-1 μL of adenovirus in culture medium; medium was replaced 12-20 h after infection. Transduction efficiency was evaluated 72-96 h post-infection.

f. RTT-Like hESC and Neuronal Differentiation

Human ESCs were cultured on irradiated mouse embryonic fibroblasts (MEFs) with standard hESCs medium: [DMEM/F12 (Invitrogen) supplemented with 15% fetal bovine serum (Gibco HI FBS, 10082-147), 5% KnockOut Serum Replacement (Invitrogen), 2 mM L-glutamine (MPBio), 1% nonessential amino acids (Invitrogen), 1% penicillin-streptomycin (Lonza), 0.1 mM β-mercaptoethanol (Sigma) and 4 ng/ml FGF2 (R&D systems)]. To differentiate hESC into neurons, 2 or 3 wells of confluent cells from 6-well plate are treated with ROCK Inhibitor overnight. Wash cells with PBS once, and then Collagenase IV (1 mg/ml) treatment for 45 min. Collect and grow the harvested hESC aggregates on a non-adherent 6-well plate for 5 days with Noggin (500 ng/ml) and SB (10 μM) in DMEM/F12 medium and then remove SB for another 2 days. For the next 7 days, treat the cells with FGF (20 ng/ml). To harvest, wash with PBS and incubate for 10 min with PBS with EDTA. Then incubate for 10 min with Accutase. Harvest to prepare droplet plate onto Poly-ornithine/Laminin coated plates and incubate in 37° incubator for 2 hours followed by adding 5 ml medium for each well. To differentiate these neuronal precursor cells into neurons, withdraw FGF and grow them on Matrigel-coated plate in DMEM/F12 for 2-4 weeks to promote maturation.

g. Whole-Cell Electrophysiology.

Whole-cell voltage-clamp recordings for HEK 293 were collected at room temperature using an Axopatch 200B amplifier (Axon Instruments). Glass pipettes were pulled with a horizontal puller (P97; Sutter Instruments Co.) and fire-polished (Microforge, Narishige, Tokyo, Japan), resulting in 2-5-megaOhm resistances, before series resistance compensation of 70%. Internal solutions in experiments performed in HEK 293 cells contained 135 mM CsMeSO₃, 5 mM CsCl₂, 1 mM MgCl₂, 4 mM MgATP, 10 mM HEPES, and 10 mM BAPTA. Internal solutions in experiments performed in cultured cardiomyocytes contained 114 mM 3, 5 mM CsCl2, 1 mM MgCl, 4 mM MgATP, 10 mM HEPES, 10 mM BAPTA, and 5 μM ryanodine. Internal solutions were adjusted to 290-295 mOsm with CsMeSO₃ and to pH 7.4 with CsOH.

For experiments measuring Ca_{V}1 currents expressed heterologously in HEK 293 cells, the external solution contained 140 mM TEA-MeSO₃, 10 mM HEPES (pH 7.4), and 40 mM BaCl₂. For experiments measuring Ca_{V}2 currents expressed heterologously in HEK 293 cells, the external solution contained 140 mM TEA-MeSO₃, 10 mM HEPES (pH 7.4), and 5 mM BaCl₂. For experiments measuring L-type currents in cultured cardiomyocytes, the external solutions contained 140 mM TEA-MeSO₃, 10 mM HEPES (pH 7.4), and 5 mM CaCl₂. External solutions were adjusted to 300 mOsm with TEA-MeSO₃ and pH 7.4 with TEA-OH. Individual cells with no adjacent contacts were selected for recordings. In HEK 293 cells, peak currents were determined by measuring steady state currents after 15 ms test pulses from −50 to +90 mV. For experiments involving cardiomyocytes, cultured cells were incubated 10 minutes prior to recordings in a bath solution containing 140 mM TEA-MeSO₃, 10 mM HEPES (pH 7.4), 5 mM CaCl₂ and 5 μM ryanodine. Currents were elicited by 300 ms depolarization at −50 to +50 mV. Custom MATLAB (Mathworks) software was used to determine peak currents.

h. Flow-Cytometric FRET 2-Hybrid Assay

HEK 293T cells were cultured in 12-well plates and transfected with polyethylenimine (Polysciences Inc.) or FuGENE 4K (Promega). LRRC10-Cer and Holo-CaV1.2 venus (Ven)-tagged cDNA pairs (20 ng and 2 μg, respectively) were mixed in serum-free DMEM media. FRET experiments were performed 3-5 days post-transfection. Cycloheximide was added to wells at a final concentration of 100 μM 2 hours before flow-cytometric evaluation to halt new fluorophore synthesis and allow for maturation of existing fluorophores. FRET measurements were done using LSR II (BD Biosciences) flow cytometer, equipped with 405-nm, 488-nm and 633-nm lasers for excitation and 18 different emission channels. Analysis was performed as described in Liu et al., Mechanism of adrenergic Ca_{V}1.2 stimulation revealed by proximity proteomics. Nature 577, 695-700 (2020).

i. Single-Channel Electrophysiology

Pipettes were pulled from ultra-thick-walled borosilicate glass (BF200-116-10, Sutter Instruments) as described in previous section, resulting in resistances of 5-10 megaOhm. To reduce noise, pipettes were distally coated with Sylgard (Dow Corning). External solution contained 132 K⁺-glutamate, 5 KCl, 5 NaCl, 3 MgCl₂, 2 EGTA, 10 glucose, 20 HEPES, at 300 mOsm adjusted with glucose, and pH 7.4 adjusted with NaOH. Internal solution contained 140 mM TEA-MeSO3, 10 mM HEPES (pH 7.4), and 40 mM BaCl2, adjusted to 300 mosM with TEA-MeSO₃ and pH 7.4 with TEA-OH. Solutions were chosen based on previous studies (67). Single channel opening was elicited during a 200 ms voltage ramp from −80 to +60 mV. For each patch, 100-200 sweeps were recorded. Number of channels per recording were then determined by obtaining additional 80-150 sweeps after addition of Bay K 8644 to the external solution (final concentration 9 μM). Patches containing up to 5 channels were analyzed.

Total and Surface Calcium Channel Flow-Cytometric Assay. Total and surface Ca²⁺ channel populations were evaluated using flow cytometry in live, HEK 293 cells as in previous studies. Briefly, transfected HEK 293 cells cultured in 12-well plates were gently washed with ice cold PBS containing Ca²⁺ and Mg²⁺ (in mM: 0.9 CaCl2, 0.49 MgCl2, pH 7.4) 72-96 hours post transfection. Cells were then blocked for 30 with DMEM containing 3% bovine serum albumin at 4° C., followed by 1 hour incubation in DMEM/3% bovine serum albumin containing 1 μM AlexaFluor 647 conjugated α-bungarotoxin (Life Technologies) at 4° C. HEK 293 cells were then rinsed thrice with PBS containing Ca²⁺ and Mg²⁺. Cells were harvested in Ca²⁺-free PBS and evaluated by flow cytometry using a BD Fortessa Cell Analyzer (BD Biosciences). Data was analyzed using FlowJo flow cytometry analysis software (BD Biosciences).

j. Isolated Neuron Stimulation.

All experiments were performed on culture day 14-16. Following a previously validated protocol, neurons were pretreated with 1 μM TTX, 10 μM APV, and 10 μM NBQX resuspended into culture medium to prevent spontaneous depolarization and block glutamate-driven neurotransmission. After 4 hours, glass coverslips were transferred to a resting solution containing 150 mM NaCl, 4 mM KCl, 2 mM MgCl2, 2 mM CaCl2, 10 mM HEPES, 10 mM glucose at pH 7.4 (balanced with NaOH). Neurons were stimulated for 3 minutes by transferring coverslips into a solution containing 114 mM NaCl, 40 mM KCl, 2 mM MgCl2, 2 mM CaCl2, 10 mM HEPES, 10 mM glucose at pH 7.4 (balanced with KOH). Resting and stimulation solutions also contained 1 μM TTX, 10 μM APV and 10 μM NBQX. Neurons were then transferred to fixing solution as described in immunocytochemistry section.

k. Immunocytochemistry.

Isolated neurons were fixed using ice-cold 4% paraformaldehyde in PBS with Ca²⁺ and Mg²⁺, 4% sucrose and 20 mM EGTA. Cells were then permeabilized with 0.1% Triton X-100 (Sigma Aldrich) and blocked for 1 hour with 10% HyClone fetal bovine serum (General Electric) in PBS with Ca²⁺ and Mg²⁺. Neurons were then incubated overnight at 4° C. in 1% bovine serum albumin in PBS with pCREB (Ser133) (87G3) Rabbit mAb (1:333, Cell Signaling, Cat No. #9198). Neurons were then thrice rinsed with PBS, followed by 1 hour incubation with secondary antibody (1 mg/mL anti-Rabbit Alexa Fluor 647, ThermoFisher) and thrice rinsing with PBS again. Coverslips were then mounted using DAPI-containing mounting solution and stored at 4° C.

l. Stereotaxic Surgery

Twelve- to fourteen-week-old C57BL/6 male mice (Jackson Laboratories, Bar Harbor, Maine) were placed on a stereotaxic surgical apparatus unit (David Kopf Instruments, Tujunga, CA). A midline incision was made on the scalp, and the head was leveled based on bregma and lambda. Mice were injected unilaterally in the dorsal hippocampus (n=2) and the mPFC (n=3) with two different viruses using a 2.5 μl 30-gauge Hamilton syringe. The right hemisphere received 400 nl injection of AAV9-pAAV [Exp]-CMV [GEECC 2.0]-mCherry and the left hemisphere received 400 nl injections of AAV9-pAAV [Exp]-CMV-EGFP control virus for both brain regions at 0.1 μl/min. Coordinates for the PFC (AP=2.0, DV=−2.3, ML=±0.3) and dorsal HPC (AP=−1.85, DV=−1.55, ML=±1.10) were based on the Allen Brain Atlas. The needle was left in place for an additional 5 minutes after the infusion to ensure that the virus was completely delivered before switching to the needle with the second virus. After 2.5 weeks of recovery, mice were given a ketamine/xylazine cocktail at 10 mg/kg and perfused with 1×PBS (pH 7.4) with heparin, followed by an infusion of 4% paraformaldehyde (pH 7.4). Brains were postfixed overnight in 4% PFA at 4° C. and were sectioned the next day using a Leica vibratome at 40 μm thickness.

m. Immunohistochemistry

Brain sections were evaluated for expression of mCherry for confirmation of AAV9-GeeC and GFP for AAV9-GFP viral infection. After confirmation of expression, tissue sections were washed 3× for 10 minutes in 1×PBS. Tissue was then blocked in a solution of 1×PBS/3% NGS/0.1% TritonX for 1 hour at room temperature, followed by 3× washes for 10 minutes in 1×PBS. Phospho-CREB (pCREB) (Ser133) (87G3) Rabbit mAb (Cell Signaling, Cat No. #9198) was diluted in 1×PBS/1% BSA/0.1% TritonX (1:1000) followed by overnight incubation at 4° C. The following day, tissue was rinsed 3× in 1×PBS for 10 minutes, and then incubated in Donkey anti-Rabbit IgG (H+L) Highly Cross-Adsorbed Secondary Antibody, Alexa Fluor 647 (1:500, Thermo Fisher, Cat No. A-31573). Tissue was rinsed 3× in 1×PBS for 10 minutes and then mounted on glass slides and coverslipped with ProLong™ Glass Antifade Mountant with NucBlue™ Stain (Invitrogen, Cat No. P36981). Slides were left to rest in the dark overnight before imaging.

n. Image Acquisition and Analysis.

Fixed cultured rat neurons were imaged using a 60× (1.3 NA) oil objective on a Nikon Ti Eclipse inverted microscope equipped with a Yokogawa CSU-X1 confocal spinning disk and an Andor Zyla sCMOS camera. ImageJ (NIH) was used to quantify signal intensity, and only neurons expressing either mCherry (for GeeC) or GFP were selected for analysis; uninfected controls were selected using DAPI signal. In each field of view, a cell-free region of interest was selected as signal background, and its mean intensity was subtracted from all regions of interest. Analysis of nuclear pCREB was done by manually drawing the nuclear regions while viewing only DAPI, GFP and RFP channels, but blinded to the pCREB color channel (647 nm). Background-subtracted mean intensity was quantified and normalized it to unstimulated condition. Brain slice images were acquired for the mCherry, GFP, and Cy5 (detection of phospho-CREB) channels using a Zeiss LSM 880 Laser Scanning Confocal Microscope. All channels, laser intensities, gains and detection ranges were kept consistent between scans. GFP [488 laser (Ex. 488, Em. 525) with detection range 500-550] was used to visualize the cells infected with the AAV9-GFP reporter control, DSRed [561 laser (Ex. 561, Em. 603, detection range 580-625)] was used to image cells infected with AAV9-GeeC-mCherry, and the Cy5 channel [633 laser (Ex. 633, Em. 695, detection range 640-750)] was used to see expression of phospho-CREB after immunohistochemistry. Images were taken as a z-Stack, with bidirectional X and 4 frame averaging. The MBS 488/561/633 filter was used.

After acquisition at 40× and 20× magnification, images were processed for quantification using Fiji/ImageJ. One z-stack from each scan was chosen and placed through automatic thresholding of the GeeC/GFP channels. Using Analyze Particles, automatic ROIs were made around the soma of each GeeC/GFP tagged neuron and added to the ROI manager. Alongside, automatic ROIs were created for the background to use as a normalizer for the corrected total cell fluorescence (CTCF). Area, integrated density and mean gray values were calculated for all preset ROIs in the respective pCREB/Cy5 channel stack. CTCF was calculated using Integrated Density−(Area*Mean background grey value), to normalize pCREB intensity values for the respective background around each ROI. An unpaired t-test was performed using GraphPad PRISM to compare CTCF of pCREB in GeeC and GFP tagged neurons. Results were considered significant with a p-value<0.05.

### REFERENCES

1. B. Hille, Ionic channels of excitable membranes (Sinauer Associates, Sunderland, MA, ed. 3rd, 2001), pp. 814.G.
2. W. Zamponi, J. Striessnig, A. Koschak, A. C. Dolphin, The Physiology, Pathology, and Pharmacology of Voltage-Gated Calcium Channels and Their Future Therapeutic Potential. Pharmacol Rev 67, 821-870 (2015).
3. F. Hofmann, V. Flockerzi, S. Kahl, J. W. Wegener, L-type Ca_{V}1.2 calcium channels: from in vitro findings to in vivo function. Physiol Rev 94, 303-326 (2014).
4. D. M. Bers, Cardiac excitation-contraction coupling. Nature 415, 198-205 (2002).
5. O. Vivas, C. M. Moreno, L. F. Santana, B. Hille, Proximal clustering between BK and Ca(V)1.3 channels promotes functional coupling and BK channel activation at low voltage. eLife 6, (2017).
6. N. V. Marrion, S. J. Tavalin, Selective activation of Ca2+-activated K+ channels by co-localized Ca2+ channels in hippocampal neurons. Nature 395, 900-905 (1998).
7. R. Dolmetsch, Excitation-transcription coupling: signaling by ion channels to the nucleus.565 Sci STKE 2003, PE4 (2003).
8. D. G. Wheeler, R. D. Groth, H. Ma, C. F. Barrett, S. F. Owen, P. Safa, R. W. Tsien, Ca(V) land Ca(V)2 channels engage distinct modes of Ca(2+) signaling to control CREB-dependent gene expression. Cell 149, 1112-1124 (2012).
9. R. E. Dolmetsch, U. Pajvani, K. Fife, J. M. Spotts, M. E. Greenberg, Signaling to the nucleus by an L-type calcium channel-calmodulin complex through the MAP kinasepathway. Science 294, 333-339 (2001).
10. A. E. West, W. G. Chen, M. B. Dalva, R. E. Dolmetsch, J. M. Kornhauser, A. J. Shaywitz,M. A. Takasu, X. Tao, M. E. Greenberg, Calcium regulation of neuronal gene expression. Proc Natl Acad Sci USA 98, 11024-11031 (2001).
11. V. Schulla, E. Renstrom, R. Feil, S. Feil, I. Franklin, A. Gjinovci, X. J. Jing, D. Laux, I. Lundquist, M. A. Magnuson, S. Obermuller, C. S. Olofsson, A. Salehi, A. Wendt, N. Klugbauer, C. B. Wollheim, P. Rorsman, F. Hofmann, Impaired insulin secretion and glucose tolerance in beta cell-selective Ca(v)1.2 Ca2+ channel null mice. The EMBOjournal 22, 3844-3854 (2003).
12. A. Marcantoni, D. H. Vandael, S. Mahapatra, V. Carabelli, M. J. Sinnegger-Brauns, J. Striessnig, E. Carbone, Loss of Ca_{V}1.3 channels reveals the critical role of L-type and BK channel coupling in pacemaking mouse adrenal chromaffin cells. J Neurosci 30, 491-504(2010).
13. D. C. Hill-Eubanks, M. E. Werner, T. J. Heppner, M. T. Nelson, Calcium signaling in smooth muscle. Cold Spring Harbor perspectives in biology 3, a004549 (2011).
14. A. Brandt, J. Striessnig, T. Moser, CaV1.3 channels are essential for development and presynaptic activity of cochlear inner hair cells. J Neurosci 23, 10832-10840 (2003).
15. G. S. Pitt, M. Matsui, C. Cao, Voltage-Gated Calcium Channels in Nonexcitable Tissues. 589 Annu Rev Physiol 83, 183-203 (2021).
16. Splawski, K. W. Timothy, L. M. Sharpe, N. Decher, P. Kumar, R. Bloise, C. Napolitano, P. J. Schwartz, R. M. Joseph, K. Condouris, H. Tager-Flusberg, S. G. Priori, M. C. Sanguinetti, M. T. Keating, Ca(V)1.2 calcium channel dysfunction causes a multi system disorder including arrhythmia and autism. Cell 119, 19-31 (2004).
17. D. Endres, N. Decher, I. Rohr, K. Vowinkel, K. Domschke, K. Komlosi, A. Tzschach, B. Glaser, M. A. Schiele, K. Runge, P. Suss, F. Schuchardt, K. Nickel, B. Stallmeyer, S. Rinne, E. Schulze-Bahr, L. Tebartz van Elst, New Ca_{V}1.2 Channelopathy with High-FunctioningAutism, Affective Disorder, Severe Dental Enamel Defects, a Short QT Interval, and a Novel CACNAIC Loss-Of-Function Mutation. International journal of molecularsciences 21, (2020).
18. C. Antzelevitch, G. D. Pollevick, J. M. Cordeiro, O. Casis, M. C. Sanguinetti, Y. Aizawa, A. Guerchicoff, R. Pfeiffer, A. Oliva, B. Wollnik, P. Gelber, E. P. Bonaros, Jr., E. Burashnikov, Y. Wu, J. D. Sargent, S. Schickel, R. Oberheiden, A. Bhatia, L. F. Hsu, M. Haissaguerre, R. Schimpf, M. Borggrefe, C. Wolpert, Loss-of-function mutations in the cardiac calcium channel underlie a new clinical entity characterized by ST-segment elevation, short QT intervals, and sudden cardiac death. Circulation 115, 442-449 (2007).
19. L. H. Rodan, R. C. Spillmann, H. T. Kurata, S. M. Lamothe, J. Maghera, R. A. Jamra, A. Alkelai, S. E. Antonarakis, I. Atallah, O. Bar-Yosef, F. Bilan, K. Bjorgo, X. Blanc, P. VanBogaert, Y. Bolkier, L. C. Burrage, B. U. Christ, J. L. Granadillo, P. Dickson, K. A. Donald, C. Dubourg, A. Eliyahu, L. Emrick, K. Engleman, M. V. Gonfiantini, J. M. Good, J. Kalser, C. Kloeckner, G. Lachmeijer, M. Macchiaiolo, F. Nicita, S. Odent, E. O'Heir, X. Ortiz-Gonzalez, M. Pacio-Miguez, M. Palomares-Bralo, L. Pena, K. Platzer, M. Quinodoz, E. Ranza, J. A. Rosenfeld, E. Roulet-Perez, A. Santani, F. Santos-Simarro, B. Pode-Shakked, C. Skraban, R. Slaugh, A. Superti-Furga, I. Thiffault, R. H. van Jaabrsveld, M. Vincent, H. G. Wang, P. Zacher, N. Undiagnosed Diseases, E. Rush, G. S. Pitt, P. Y. B. Au, V. Shashi, Phenotypic expansion of CACNAIC-associated disorders to include isolated neurological manifestations. Genet Med 23, 1922-1932 (2021).
20. Z. D. Kabir, A. Martinez-Rivera, A. M. Rajadhyaksha, From Gene to Behavior: L-Type Calcium Channel Mechanisms Underlying Neuropsychiatric Symptoms. Neurotherapeutics 14, 588-613 (2017).
21. S. Heyes, W. S. Pratt, E. Rees, S. Dahimene, L. Ferron, M. J. Owen, A. C. Dolphin, Genetic disruption of voltage-gated calcium channels in psychiatric and neurological disorders. Prog Neurobiol 134, 36-54 (2015).
22. C. International Schizophrenia, S. M. Purcell, N. R. Wray, J. L. Stone, P. M. Visscher, M. C. O'Donovan, P. F. Sullivan, P. Sklar, Common polygenic variation contributes to risk of schizophrenia and bipolar disorder. Nature 460, 748-752 (2009).
23. C. Cross-Disorder Group of the Psychiatric Genomics, Identification of risk loci with shared effects on five major psychiatric disorders: a genome-wide analysis. Lancet 381, 1371-1379 (2013).
24. S. A. Ament, S. Szelinger, G. Glusman, J. Ashworth, L. Hou, N. Akula, T. Shekhtman, J. A. Badner, M. E. Brunkow, D. E. Mauldin, A. B. Stittrich, K. Rouleau, S. D. Detera-Wadleigh, J. I. Nurnberger, Jr., H. J. Edenberg, E. S. Gershon, N. Schork, S. BipolarGenome, N. D. Price, R. Gelinas, L. Hood, D. Craig, F. J. McMahon, J. R. Kelsoe, J. C. Roach, Rare variants in neuronal excitability genes influence risk for bipolar disorder. Proc Natl Acad Sci USA 112, 3576-3581 (2015).
25. A. Pinggera, J. Striessnig, Ca(v) 1.3 (CACNAID) L-type Ca(2+) channel dysfunction in CNS disorders. J Physiol 594, 5839-5849 (2016).
26. G. H. Hockerman, B. Z. Peterson, B. D. Johnson, W. A. Catterall, Molecular determinants of drug binding and action on L-type calcium channels. Annual review of pharmacology and toxicology 37, 361-396 (1997).
27. T. Yang, Y. Suhail, S. Dalton, T. Kernan, H. M. Colecraft, Genetically encoded molecules for inducibly inactivating Ca_{V} channels. Nature chemical biology 3, 795-804 (2007)
28. S. A. Kanner, T. Morgenstern, H. M. Colecraft, Sculpting ion channel functional expressionwith engineered ubiquitin ligases. eLife 6, (2017).
29. A. Bourson, P. C. Moser, A. J. Gower, A. K. Mir, Central and peripheral effects of the dihydropyridine calcium channel activator BAY K 8644 in the rat. European journal of pharmacology 160, 339-347 (1989).
30. R. C. Shelton, J. A. Grebb, W. J. Freed, Induction of seizures in mice by intracerebroventricular administration of the calcium channel agonist BAY k 8644. Brain research 402, 399-402 (1987).
31. H. A. Jinnah, S. Yitta, T. Drew, B. S. Kim, J. E. Visser, J. D. Rothstein, Calcium channel activation and self-biting in mice. Proc Natl Acad Sci USA 96, 15228-15232 (1999).
32. H. Chen, D. H. Vandorpe, X. Xie, S. L. Alper, M. L. Zeidel, W. Yu, Disruption of Cav1.2-mediated signaling is a pathway for ketamine-induced pathology. Nature communications 11, 4328 (2020).
33. W. A. Catterall, M. J. Lenaeus, T. M. Gamal El-Din, Structure and Pharmacology of Voltage-Gated Sodium and Calcium Channels. Annual review of pharmacology and toxicology 60, 133-154 (2020).
34. G. Liu, A. Papa, A. N. Katchman, S. I. Zakharov, D. Roybal, J. A. Hennessey, J. Kushner, L. Yang, B. X. Chen, A. Kushnir, K. Dangas, S. P. Gygi, G. S. Pitt, H. M. Colecraft, M. Ben-Johny, M. Kalocsay, S. O. Marx, Mechanism of adrenergic Ca_{V}1.2 stimulation revealed by proximity proteomics. Nature 577, 695-700 (2020).
35. M. Ben-Johny, D. T. Yue, Calmodulin regulation (calmodulation) of voltage-gated calcium channels. J Gen Physiol 143, 679-692 (2014).
36. M. T. Woon, P. A. Long, L. Reilly, J. M. Evans, A. M. Keefe, M. R. Lea, C. J. Beglinger, R. C. Balijepalli, Y. Lee, T. M. Olson, T. J. Kamp, Pediatric Dilated Cardiomyopathy-Associated LRRC10 (Leucine-Rich Repeat-Containing 10) Variant Reveals LRRC10 as an Auxiliary Subunit of Cardiac L-Type Ca(2+) Channels. J Am Heart Assoc 7, (2018).
37. N. L. Manuylov, E. Manuylova, V. Avdoshina, S. Tevosian, Serdin1/Lrrc10 is dispensable for mouse development. Genesis 46, 441-446 (2008).
38. P. D. Nguyen, I. Gooijers, G. Campostrini, A. O. Verkerk, H. Honkoop, M. Bouwman, D. E. M. de Bakker, T. Koopmans, A. Vink, G. E. M. Lamers, A. Shakked, J. Mars, A. A. Mulder, S. Chocron, K. Bartscherer, E. Tzahor, C. L. Mummery, T. P. de Boer, M. Bellin, J. Bakkers, Interplay between calcium and sarcomeres directs cardiomyocyte maturation during regeneration. Science 380, 758-764 (2023).
39. W. T. Stockdale, M. E. Lemieux, A. C. Killen, J. Zhao, Z. Hu, J. Riepsaame, N. Hamilton, T. Kudoh, P. R. Riley, R. van Aerle, Y. Yamamoto, M. T. M. Mommersteeg, Heart Regeneration in the Mexican Cavefish. Cell Rep 25, 1997-2007 e1997 (2018).
40. K. H. Kim, D. S. Antkiewicz, L. Yan, K. W. Eliceiri, W. Heideman, R. E. Peterson, Y. Lee, Lrrc10 is required for early heart development and function in zebrafish. Dev Biol 308, 494-506 (2007).
41. R. J. Salamon, M. C. Mckeon, J. Bae, X. Zhang, W. G. Paltzer, K. N. Wanless, A. R. Schuett, D. J. Nuttall, S. A. Nemr, R. Sridharan, Y. Lee, T. J. Kamp, A. I. Mahmoud, LRRC10 regulates mammalian cardiomyocyte cell cycle during heart regeneration. NPJ Regen Med 8, 39 (2023).
42. M. J. Brody, L. Feng, A. C. Grimes, T. A. Hacker, T. M. Olson, T. J. Kamp, R. C. Balijepalli, Y. Lee, LRRC10 is required to maintain cardiac function in response to pressure overload. Am J Physiol Heart Circ Physiol 310, H269-278 (2016).
43. P. Enkhbayar, M. Kamiya, M. Osaki, T. Matsumoto, N. Matsushima, Structural principles of leucine-rich repeat (LRR) proteins. Proteins 54, 394-403 (2004).
44. N. Matsushima, S. Takatsuka, H. Miyashita, R. H. Kretsinger, Leucine Rich Repeat Proteins: Sequences, Mutations, Structures and Diseases. Protein Pept Lett 26, 108-131 (2019).
45. T. J. Morgenstern, J. Park, Q. R. Fan, H. M. Colecraft, A potent voltage-gated calcium channel inhibitor engineered from a nanobody targeted to auxiliary Ca(V) beta subunits. Elife 8, (2019).
46. Z. Buraei, J. Yang, The beta subunit of voltage-gated Ca2+ channels. Physiol Rev 90, 1461-1506 (2010).
47. E. Perez-Reyes, Molecular physiology of low-voltage-activated t-type calcium channels. Physiol Rev 83, 117-161 (2003).
48. T. Yang, X. Xu, T. Kernan, V. Wu, H. M. Colecraft, Rem, a member of the RGK GTPases, inhibits recombinant Ca_{V}1.2 channels using multiple mechanisms that require distinct conformations of the GTPase. J Physiol 588, 1665-1681 (2010).
49. B. Li, M. R. Tadross, R. W. Tsien, Sequential ionic and conformational signaling by calcium channels drives neuronal gene expression. Science 351, 863-867 (2016).
50. D. G. Wheeler, C. F. Barrett, R. D. Groth, P. Safa, R. W. Tsien, CaMKII locally encodes L-type channel activity to signal to nuclear CREB in excitation-transcription coupling. J Cell Biol 183, 849-863 (2008).
51. J. P. K. Ip, N. Mellios, M. Sur, Rett syndrome: insights into genetic, molecular and circuit mechanisms. Nature Reviews 19, 368-382 (2018).
52. W. G. Chen, Q. Chang, Y. Lin, A. Meissner, A. E. West, E. C. Griffith, R. Jaenisch, M. E. Greenberg, Derepression of BDNF transcription involves calcium-dependent phosphorylation of MeCP2. Science 302, 885-889 (2003).
53. Z. Zhou, E. J. Hong, S. Cohen, W. N. Zhao, H. Y. Ho, L. Schmidt, W. G. Chen, Y. Lin, E. Savner, E. C. Griffith, L. Hu, J. A. Steen, C. J. Weitz, M. E. Greenberg, Brain-specific phosphorylation of MeCP2 regulates activity-dependent Bdnf transcription, dendritic growth, and spine maturation. Neuron 52, 255-269 (2006).
54. N. Ballas, D. T. Lioy, C. Grunseich, G. Mandel, Non-cell autonomous influence of MeCP2-deficient glia on neuronal dendritic morphology. Nat Neurosci 12, 311-317 (2009).
55. Y. Li, H. Wang, J. Muffat, A. W. Cheng, D. A. Orlando, J. Loven, S. M. Kwok, D. A. Feldman, H. S. Bateup, Q. Gao, D. Hockemeyer, M. Mitalipova, C. A. Lewis, M. G. Vander Heiden, M. Sur, R. A. Young, R. Jaenisch, Global transcriptional and translational repression in human-embryonic-stem-cell-derived Rett syndrome neurons. Cell Stem Cell 13, 446-458 (2013).
56. J. Qian, X. Guan, B. Xie, C. Xu, J. Niu, X. Tang, C. H. Li, H. M. Colecraft, R. Jaenisch, X. S. Liul, Multiplex epigenome editing of MECP2 to rescue Rett syndrome neurons. Science Translational Medicine 15, (2023).
57. W. Yu, Reviving Ca_{V}1.2 as an attractive drug target to treat bladder dysfunction. FASEB J36, e22118 (2022).
58. I. F. Khan, R. K. Hirata, D. W. Russell, AAV-mediated gene targeting methods for human cells. Nature protocols 6, 482-501 (2011).
59. Q. Bu, A. Wang, H. Hamzah, A. Waldman, K. Jiang, Q. Dong, R. Li, J. Kim, D. Turner, Q. Chang, CREB Signaling Is Involved in Rett Syndrome Pathogenesis. J Neurosci 37, 3671-3685 (2017).
60. S. Kamijo, Y. Ishii, S. I. Horigane, K. Suzuki, M. Ohkura, J. Nakai, H. Fujii, S. Takemoto-Kimura, H. Bito, A Critical Neurodevelopmental Role for L-Type Voltage-Gated Calcium Channels in Neurite Extension and Radial Migration. J Neurosci 38, 5551-5566 (2018).
61. J. N. Muth, I. Bodi, W. Lewis, G. Varadi, A. Schwartz, A Ca(2+)-dependent transgenic model of cardiac hypertrophy: A role for protein kinase Calpha. Circulation 103, 140-147 (2001).
62. B. M. Ahern, B. M. Levitan, S. Veeranki, M. Shah, N. Ali, A. Sebastian, W. Su, M. C. Gong, J. Li, J. E. Stelzer, D. A. Andres, J. Satin, Myocardial-restricted ablation of the GTPase RAD results in a pro-adaptive heart response in mice. J Biol Chem 294, 10913-10927 (2019).
63. M. J. Brody, Y. Lee, The Role of Leucine-Rich Repeat Containing Protein 10 (LRRC10) in Dilated Cardiomyopathy. Front Physiol 7, 337 (2016).
64. M. J. Brody, T. A. Hacker, J. R. Patel, L. Feng, J. Sadoshima, S. G. Tevosian, R. C. Balijepalli, R. L. Moss, Y. Lee, Ablation of the cardiac-specific gene leucine-rich repeat containing 10 (Lrrc10) results in dilated cardiomyopathy. PloS one 7, e51621 (2012).
65. X. K. Qu, F. Yuan, R. G. Li, L. Xu, W. F. Jing, H. Liu, Y. J. Xu, M. Zhang, X. Liu, W. Y. Fang, Y. Q. Yang, X. B. Qiu, Prevalence and spectrum of LRRC10 mutations associated with idiopathic dilated cardiomyopathy. Mol Med Rep 12, 3718-3724 (2015).
66. L. Huang, S. Tang, Y. Chen, L. Zhang, K. Yin, Y. Wu, J. Zheng, Q. Wu, J. C. Makielski, J. Cheng, Molecular pathological study on LRRC10 in sudden unexplained nocturnal death syndrome in the Chinese Han population. Int J Legal Med 131, 621-628 (2017).
67. P. J. Adams, M. Ben-Johny, I. E. Dick, T. Inoue, D. T. Yue, Apocalmodulin itself promotes ion channel opening and Ca(2+) regulation. Cell 159, 608-622 (2014).
68. J. Niu, I. E. Dick, W. Yang, M. A. Bamgboye, D. T. Yue, G. Tomaselli, T. Inoue, M. Ben-Johny, Allosteric regulators selectively prevent Ca(2+)-feedback of Ca(V) and Na(V)channels. eLife 7, (2018).
69 B. Z. Stanton, E. J. Chory, G. R. Crabtree, Chemically induced proximity in biology and medicine. Science 359, (2018).
70. J. Niu, M. Ben Johny, I. E. Dick, T. Inoue, Following Optogenetic Dimerizers and Quantitative Prospects. Biophys J 111, 1132-1140 (2016).
71. G. G. Gross, J. A. Junge, R. J. Mora, H. B. Kwon, C. A. Olson, T. T. Takahashi, E. R. Liman, G. C. Ellis-Davies, A. W. McGee, B. L. Sabatini, R. W. Roberts, D. B. Arnold, Recombinant probes for visualizing endogenous synaptic proteins in living neurons. Neuron 78, 971-985 (2013).
72. S. Kanner, T. J. Morgenstern, H. M. Colecraft, Sculpting ion channel functional expression with engineered ubiquitin ligases. eLife In press, (2017).
73. N. Chakouri, S. Rivas, D. Roybal, L. Yang, J. Diaz, A. Hsu, R. Mahling, B. X. Chen, J. O. Owoyemi, D. DiSilvestre, D. Sirabella, B. Corneo, G. F. Tomaselli, I. E. Dick, S. O. Marx, M. Ben-Johny, Fibroblast growth factor homologous factors serve as a molecular rheostatin tuning arrhythmogenic cardiac late sodium current. Nat Cardiovasc Res 1, 1-13 (2022).
74. K. Deisseroth, E. K. Heist, R. W. Tsien, Translocation of calmodulin to the nucleus supports CREB phosphorylation in hippocampal neurons. Nature 392, 198-202 (1998).
75. B. Li, M. R. Tadross, R. W. Tsien, Sequential ionic and conformational signaling by calcium channels drives neuronal gene expression. Science 351, 863-867 (2016).

What is claimed:

1. A fusion protein, wherein the fusion protein comprises:
a polypeptide derived from leucine-rich repeat-containing protein 10 (LRRC10); and
a targeting agent,
wherein:
the targeting agent is conjugated to the polypeptide;
the targeting agent is selective for a β subunit of a L-type Ca²⁺ channel (LTCC); and
the polypeptide modulates channel activity of Ca_{V}1.2 and/or Ca_{V}1.3.

2. The fusion protein of claim 1, wherein the polypeptide enhances channel activity of Ca_{V}1.2 and/or Ca_{V}1.3.

3. The fusion protein of claim 1, wherein the polypeptide comprises the amino-terminus (NT domain) of LRRC10 or a portion thereof.

4. The fusion protein of claim 1, wherein the polypeptide comprises the first 53 amino acids of LRRC10.

5. The fusion protein of claim 1, wherein the polypeptide has an amino acid sequence comprising less than 60 amino acids.

6. The fusion protein of claim 1, wherein:
the targeting agent has a greater binding affinity to the β subunit of LTCC than the polypeptide; and/or
the LTCC is Ca_{V}1.2 or Ca_{V}1.3; and/or
the targeting agent is a nanobody.

7. The fusion protein of claim 6, wherein the nanobody is nb.F3.

8. The fusion protein of claim 1, wherein the polypeptide is derived from human LRRC10.

9. The fusion protein of claim 1, wherein:
the amino acid sequence of the fusion protein is set forth in SEQ ID NO. 5; or
the nucleic acid sequence encoding the fusion protein is set forth in SEQ ID NO. 6.

10. A nucleic acid sequence encoding the fusion protein of claim 1.

11. A pharmaceutical composition comprising the nucleic acid sequence of claim 10.

12. The pharmaceutical composition of claim 11, wherein the nucleic acid sequence encoding the fusion protein is set forth in SEQ ID NO. 6.

13. A pharmaceutical composition comprising:
an adenovirus vector, wherein the adenovirus vector comprises the nucleic acid sequence of claim 10; or
an adenovirus generated using an adenovirus vector comprising the nucleic acid sequence of claim 10.

14. A method of upregulating Ca_{V}1.2 and/or Ca_{V}1.3 current in a cell or enhancing L-type currents in a cell, the method comprising:
administering to the cell a fusion protein comprising:
a polypeptide derived from leucine-rich repeat-containing protein 10 (LRRC10); and
a targeting agent,
wherein:
the targeting agent is conjugated to the polypeptide;
the targeting agent is selective for a β subunit of a L-type Ca²⁺ channel (LTCC); and
the polypeptide comprises the amino-terminus (NT domain) of LRRC10 or a portion thereof.

15. The method of claim 14, wherein the cell is a neuron or a muscle cell, optionally, wherein the muscle cell is a cardiomyocyte.

16. The method of claim 14, wherein the fusion protein is administered to the cell via transducing the cell with an adenovirus, the adenovirus comprising a nucleic acid sequence encoding the fusion protein.

17. A method of reducing neurological symptoms in a subject diagnosed with Rett Syndrome (RTT), the method comprising:
administering to the subject a fusion protein comprising:
a polypeptide derived from leucine-rich repeat-containing protein 10 (LRRC10); and
a targeting agent,
wherein:
the targeting agent is conjugated to the polypeptide;
the targeting agent is selective for a β subunit of a L-type Ca²⁺ channel (LTCC); and
the polypeptide comprises the amino-terminus (NT domain) of LRRC10 or a portion thereof.

18. The method of claim 17, wherein the subject is administered the fusion protein orally.

19. The method of claim 17, wherein the subject is administered the fusion protein by intravenous, intraarterial, intramuscular, intraperitoneal, subcutaneous, intradermal, intraosseous, intracardiac, intraarticular, intracavernous, periarticular, or intracranial injection.

20. The method of claim 19, wherein the fusion protein is administered by injecting the subject with an adenovirus, the adenovirus comprising a nucleic acid sequence encoding the fusion protein.
